(19) United States
(12) Patent Application Publication
(10) Pub. No.: US 2025/0032201 A1
(43) Pub. Date: Jan. 30, 2025

(54) BEDSIDE ROBOTICS DRIVE SYSTEM

(71) Applicant: Imperative Care, Inc., Campbell, CA (US)

(72) Inventors: Kyle Bartholomew, Campbell, CA (US); Matthew Hutter, Los Angeles, CA (US); Hilary Ann Koster, Easley, SC (US); Roman Devegenzo, San Jose, CA (US)

(21) Appl. No.: 18/784,603

(22) Filed: Jul. 25, 2024

Related U.S. Application Data
(60) Provisional application No. 63/529,115, filed on Jul. 26, 2023.

(51) Int. Cl. A61B 34/30; A61B 17/34; A61B 90/00; A61G 13/10; A61M 25/00; A61M 25/01; A61M 25/09

(52) U.S. Cl. CPC A61B 34/30; A61B 17/3498; A61G 13/101; A61B 2034/301; A61B 2034/303; A61B 2090/3933; A61B 2217/005; A61B 2217/007; A61M 25/0097; A61M 25/0113; A61M 25/09041

(57) ABSTRACT

A robotic medical system for performing a vascular procedure includes a procedure rail having a drive surface oriented along a generally vertical plane, a rail adjustment system configured to couple to a patient support table and having one or more movable arm segments, a fluidics system, and a plurality of catheter hubs operatively coupled to the procedure rail and configured to be axially translated along the drive surface along a longitudinal axis of the procedure rail. At least one of the plurality of catheter hubs is in fluid communication with the fluidics system, the at least one of the plurality of catheter hubs having a hemostasis valve and a catheter in communication with the hemostasis valve.

### INCORPORATION BY REFERENCE TO ANY PRIORITY APPLICATIONS

Any and all applications for which a foreign or domestic priority claim is identified in the Application Data Sheet filed with the present application are hereby incorporated by reference under 37 C.F.R. § 1.57. The present application claims priority to U.S. Provisional Patent Application No. 63/529,115, filed Jul. 26, 2023, tilted BEDSIDE ROBOTICS DRIVE SYSTEM, the entire content of which is incorporated by reference herein for all purposes and forms a part of this specification.

### BACKGROUND

### Field

The present application relates to neurovascular procedures, and more particularly, to catheter assemblies and robotic control systems for neurovascular site access.

### Description of the Related Art

A variety of neurovascular procedures can be accomplished via a transvascular access, including thrombectomy, diagnostic angiography, embolic coil deployment, and stent placement. However, the delivery of neurovascular care is limited or delayed by a variety of challenges. For example, there are not enough trained interventionalists and centers to meet the current demand for neuro interventions. Neuro interventions are difficult, with complex set up requirements and demands on the surgeon's dexterity. With two hands, the surgeon must exert precise control over 3-4 coaxial catheters plus manage the fluoroscopy system and patient position.

Long, tortuous anatomy, requires delicate, precise maneuvers. Inadvertent catheter motion can occur due to energy storage and release caused by frictional interplay between coaxial shafts and the patient's vasculature. Supra-aortic access necessary to reach the neurovasculature is challenging to achieve, especially Type III arches. Once supra-aortic access is achieved, adapting the system for neurovascular treatments is time consuming and requires guidewire and access catheter removal and addition of a procedure catheter (and possibly one or more additional catheters) to the stack.

Further, draping for the segregation of sterile and non-sterile zones is cumbersome and time consuming, especially with robotic healthcare devices. Other considerations with medical devices are the size and footprint, since space, especially in an operating room, can be limited.

During the course of a procedure, multiple different fluids and/or fluid volumes may be injected at different times in addition to aspiration. As such, fluid sources such as syringes are frequently connected and disconnected from the Luer connection port. This conventional switching of components, syringes, and fluidic connections during a procedure can lead to a risk of air bubble introduction, errors at connection points, and/or errors in fluid selection.

Thus, there remains a need for a supra-aortic access system that addresses some or all of these challenges and increases the availability of neurovascular procedures. Preferably, the system is additionally capable of driving devices further distally through the supra-aortic access to accomplish procedures in the intracranial vessels. Additionally, there remains a need for an improved fluid and tool management system that overcomes one or more of the drawbacks of conventional fluid management and catheter exchange systems.

### SUMMARY

In some aspects, the techniques described herein relate to a robotic device drive system including: a plurality of robotic arms arranged on a rail and configured to be axially translated along the rail from a contracted configuration to an expanded configuration, wherein at least one of the plurality of robotic arms include: a fluidics system configured to be fluidly coupled to a hemostasis valve; and a catheter hub coupled to a catheter, the catheter hub including the hemostasis valve, wherein axial translation of the at least one of the plurality of robotic arms causes axial translation of the catheter.

In some aspects, the techniques described herein relate to a robotic device drive control system including: a robotic arm configured to move axially along a rail, the robotic arm including: a catheter hub coupled to a catheter and configured to roll the catheter; and a valve system in communication with the catheter hub and configured to administer fluids to a hemostasis valve coupled to a portion of the catheter.

In some aspects, the techniques described herein relate to a robotic device drive control system including: at least one processor; and a robotic arm including a catheter hub and a valve system in fluid communication with the catheter hub, the catheter hub including a hemostasis valve and the at least one processor configured to execute instructions to: move the robotic arm axially along a rail of a patient support device, roll a catheter coupled to the hemostasis valve of the catheter hub, and administer fluids through the valve system in fluid communication with the hemostasis valve.

In some aspects, the techniques described herein relate to a robotic device drive control system including: at least one robotic arm operatively coupled to a rail, the at least one robotic arm including a fluidics system and a catheter hub; a controller configured to be in electrical communication with the fluidics system and the catheter hub; an adapter configured to couple both the fluidics system and the catheter hub to at least one catheter, wherein the adapter enables manipulation of the at least one catheter based on signals received from the controller.

In some aspects, the techniques described herein relate to a robotic device drive control system including: a first robotic arm including a first catheter hub and a first valve system in communication with the first catheter hub; a second robotic arm including a second catheter hub and a second valve system in communication with the second catheter hub; and a rail, wherein the first robotic arm and second robotic arm are operatively coupled to the rail, wherein the first catheter hub is configured to be in communication with a first hemostasis valve through a first interface, the first interface being configured to: receive movement control signals to move a first catheter associated with the first hemostasis valve, and receive fluid control signals for administering fluid from the first valve system to the first catheter.

In some aspects, the techniques described herein relate to a robotic device drive control system including: a rail; a rail adjustment system configured to manipulate a position of the rail between a stored configuration to a procedure configuration; a fluidics system; and a plurality of catheter hubs operatively coupled to the rail and configured to be axially translated along a longitudinal axis of the rail, wherein at least one of the plurality of catheter hubs is in fluid communication with the fluidics system, the at least one of the plurality of catheter hubs comprising a hemostasis valve and a catheter in communication with the hemostasis valve.

The fluidics system can be configured to selectively supply or vacuum to the hemostasis valve. The fluidics system can be configured to selectively supply fluid from a saline source and a contrast source.

In some aspects, the techniques described herein relate to a method of performing thrombectomy with a robotic device drive system, the method including: translating a first robotic arm operatively coupled to a first catheter axially along a rail and toward an access point vasculature of a patient to introduce the first catheter into the access point vasculature of the patient; translating a second robotic arm operatively coupled to a second catheter positioned concentrically within a lumen of the first catheter axially along the rail; and selectively supplying one or more fluids to or aspirating fluids from to one or both of the first catheter and the second catheter by a fluidics system.

In some aspects, the techniques described herein relate to a method of performing a thrombectomy procedure with a robotic device system, the method including: positioning a rail to approximately align a device stack profile to an access point vasculature of a patient; translating a first hub operatively coupled to a first catheter axially along the rail and toward the access point vasculature to introduce the first catheter into the access point vasculature; translating axially along the rail a second hub operatively coupled to a second catheter positioned within a first lumen of the first catheter; and selectively supplying one or more fluids to or aspirating fluids from one or both of: the first catheter or the second catheter by a fluidics system.

In some aspects, there is provided a robotic medical system for performing a vascular procedure. The robotic medical system includes a procedure rail having a drive surface oriented along a generally vertical plane, a rail adjustment system configured to couple to a patient support table and including one or more movable arm segments, the rail adjustment system configured to manipulate a position of the procedure rail relative to the patient support table, a fluidics system, and a plurality of catheter hubs operatively coupled to the procedure rail and configured to be axially translated along the drive surface along a longitudinal axis of the procedure rail. At least one of the plurality of catheter hubs is in fluid communication with the fluidics system, the at least one of the plurality of catheter hubs having a hemostasis valve and a catheter in communication with the hemostasis valve.

The fluidics system can be configured to selectively supply fluid or vacuum to the hemostasis valve. The fluidics system can be configured to selectively supply saline from a saline source and contrast from a contrast source to the hemostasis valve. The fluidics system can include a cassette configured to receive saline from a saline source, receive contrast from a contrast source, and receive vacuum from a vacuum source. The cassette can include one or more robotically actuated valves configured to be controlled by a control system. The robotic medical system can include a primary fluid line between the fluidics system and a junction point and a plurality of secondary fluid lines extending from the junction point, each of the plurality of secondary fluid lines coupled with one of the plurality of catheter hubs to supply fluid or vacuum thereto. The junction point can be coupled to the procedure rail. The plurality of catheter hubs can include at least one guidewire hub, at least one guide catheter hub, at least one access catheter hub, and at least one procedure catheter hub. When in a procedure position, a proximal end of the procedure rail can be positioned vertically above a distal end of the procedure rail, the distal end of the procedure rail being closer to a patient access point than the proximal end of the procedure rail. The procedure rail can be moveable from a retracted position to an extended position, wherein a distal end of the procedure rail is positioned closer to a patient access point in the extended position.

There is also be provided a method of performing a vascular procedure with a robotic medical system. The method includes positioning a procedure rail via a rail adjustment system to approximately align a device stack profile to an access point vasculature of a patient, the procedure rail having a drive surface oriented along a generally vertical plane, the rail adjustment system coupled to a patient support table and having one or more movable arm segments, translating a first hub operatively coupled to a first catheter axially along the drive surface of the procedure rail and toward the access point vasculature to introduce the first catheter into the access point vasculature, translating a second hub operatively coupled to a second catheter axially along the drive surface of the procedure rail, the second catheter positioned within a first lumen of the first catheter, and selectively supplying one or more fluids to or aspirating fluids from one or both of the first catheter and the second catheter by a fluidics system.

The method can include translating a third hub operatively coupled to a third catheter axially along the drive surface of the procedure rail, the third catheter positioned within a second lumen of the second catheter, and selectively supplying one or more fluids to or aspirating fluids from one or more of the first catheter, the second catheter, and the third catheter by the fluidics system. The method can include translating a fourth hub axially along the drive surface of the procedure rail, the fourth hub operatively coupled to a guidewire positioned within a third lumen of the third catheter. The method can include translating a third hub axially along the drive surface of the procedure rail, the third hub operatively coupled to a guidewire positioned within a second lumen of the second catheter. Selectively supplying one or more fluids to or aspirating fluids from one or both of the first catheter and the second catheter by the fluidics system can include selectively supplying saline from a saline source and contrast from a contrast source to one or both of the first catheter and the second catheter. The fluidics system can include a cassette configured to receive saline from a saline source, receive contrast from a contrast source, and receive vacuum from a vacuum source. The method can include actuating one or more robotically actuated valves by a control system. The robotic medical system can include a primary fluid line between the fluidics system and a junction point, and a first secondary fluid line extending from the junction point to the first hub to supply fluid and vacuum thereto and a second secondary fluid line extending from the junction point to the second hub to supply fluid or vacuum thereto. The junction point can be coupled to the procedure rail. When the device stack profile is approximately aligned with the patient access point, a proximal end of the procedure rail can be positioned vertically above a distal end of the procedure rail, the distal end of the procedure rail being closer to a patient access point than the proximal end of the procedure rail.

### BRIEF DESCRIPTION OF THE DRAWINGS

The above-mentioned aspects, as well as other aspects, features, and advantages of the present technology are described below in connection with various embodiments, with reference made to the accompanying drawings.

FIG. 1 illustrates an embodiment of a robotic device drive system in a stowed position.

FIG. 2 illustrates an embodiment of an embodiment of a robotic device drive system in a deployed position.

FIG. 3 illustrates a rear view of an embodiment of a robotic device drive system showing the boundaries of a sterile zone and a non-sterile zone.

FIG. 4 illustrates a rear view of an embodiment of a robotic device drive system showing a cross-section of a sterile cover for at least a portion of the robotic device drive system.

FIG. 5 illustrates arear view of an embodiment of a robotic device drive system with respect to an end of a patient support device, illustrating the vertical and horizontal adjustment of one or more arms.

FIG. 6 illustrates an embodiment of robotic device drive system in a deployed position, further illustrating the vertical and horizontal adjustment of one or more arms.

FIG. 7 illustrates an embodiment of a robotic device drive system in a deployed position, further illustrating an adjustment of one or more arms in a direction parallel to a longitudinal length of a patient support device.

FIG. 8 illustrates an embodiment of a robotic device drive system with a cross-sectional view of a catheter assembly profile passing through each arm.

FIG. 9 illustrates an embodiment of a robotic device drive system in a partially deployed or intermediate position with a catheter assembly profile passing through each deployed arm.

FIG. 10 illustrates an embodiment of a robotic device drive system in a partially deployed or intermediate position with a catheter assembly profile passing through each deployed arm.

FIG. 11 illustrates an embodiment of a distal most face of an arm including an interface adapter.

FIG. 12 illustrates an embodiment of the robotic device drive system with fluid reservoirs on three arms.

FIG. 13 illustrates an embodiment of a robotic device drive system with an overhead support structure.

FIG. 14 illustrates an embodiment of a draping barrier of a device for the embodiment of FIG. 13.

FIG. 15 illustrates an embodiment of a user interface panel for a device arm.

FIG. 16 illustrates an embodiment of various internal and external systems of a robotic device drive system.

FIG. 17 illustrates an embodiment of a fluidic system of a device arm.

FIG. 18 illustrates an embodiment of a robotic device drive system with an auxiliary rail.

FIG. 19 illustrates an embodiment of a standalone fluidics system to be used with embodiments of a robotic device drive system.

FIG. 20 illustrates a perspective view of an embodiment of a robotic device drive system with a rail adjustment system.

FIG. 21 illustrates a perspective view of the embodiment of FIG. 20.

FIG. 22 illustrates a top down view of the embodiment of FIG. 20.

FIG. 23 illustrates a side view of the embodiment of FIG. 20 with the rail in an extended position.

FIG. 24 illustrates a side view of the embodiment of FIG. 20 with the rail in a retracted position.

FIG. 25 illustrates a side view of an embodiment of a robotic device drive system with a standalone fluidics system.

FIG. 26 illustrates the sterile zone with respect to a robotic device drive system embodiment.

FIG. 27 illustrates a perspective view of an embodiment of a robotic arm of a robotic device drive system.

FIG. 28 illustrates an embodiment of a robotic device drive system in an expanded configuration.

FIG. 29 illustrates an embodiment of a robotic device drive system in a contracted configuration.

FIG. 30 illustrates an embodiment of a dual-assembly robotic device drive system in an expanded configuration.

FIG. 31 illustrates an embodiment of a robotic device drive system in an expanded configuration.

FIG. 32 illustrates a schematic of an optional standalone fluidics system to be used with embodiments of a robotic device drive system.

FIG. 33 illustrates a schematic elevational cross section through a hub adapter having a drive magnet separated from an interventional device hub and driven magnet by a sterile barrier.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Disclosed herein are systems and methods for robotic systems with standalone and integrated fluidics management systems. Reference to a fluidics system may be interpreted to specify a fluidics system integrated into a robotic arm or multiple robotic arms of robotic systems as described herein, a standalone fluidics system as described herein, or a combination of the aforementioned.

The various embodiments described herein provide systems for advancing and retracting one or more procedure or interventional devices, for example one or more catheters and/or a guidewire, into a patient's vasculature for performing various intravascular procedures. For example, various procedures may include, but are not limited to: aspiration, thrombectomy, diagnostic, endoscopic, biopsy, etc. In some implementations, these procedures (some requiring supra-aortic vessel access) may be robotic, with further embodiments having remote-control operations.

A side mounted robotic system can be positioned alongside, above, or near the patient, and configured to axially advance, retract, and in some cases rotate and/or laterally deflect one or two or three or more (e.g., concentrically, side by side oriented, rapid exchange configuration, etc.) intravascular devices. In certain implementations, each interventional device has a proximal end attached to a hub. Alternatively, in certain implementations, two or more interventional devices may be attached to a hub at a proximal end of each interventional device.

In certain embodiments, each hub can be mechanically coupled to a corresponding robotic arm. The robotic arms may translate along a patient support device to axially advance and retract one or more interventional devices (in some instances to gain supra-aortic vessel access). Additional functionality contained in a robotic arm when it is attached to a hub, attached to the interventional device, may further enable rotation and/or deflection or articulation of a distal end of the interventional device, among other optional functionality described elsewhere herein. Manipulating the interventional device hubs (e.g., via a robotic arm), in turn manipulating the interventional devices, in the described degrees of freedom can provide advancement of the interventional device, retraction of the interventional device, and the execution of procedures, e.g., clot engagement, by the interventional device. Various features and configurations of the hubs are described in U.S. patent application Ser. No. 17/527,393, filed Nov. 16, 2021, and U.S. patent application Ser. No. 18/060,935, filed Dec. 1, 2022, each of which is herein incorporated by reference in its entirety.

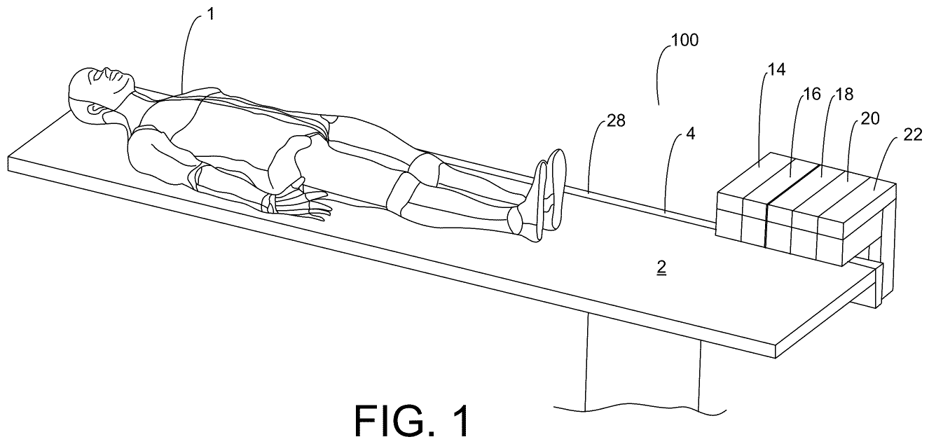

FIGS. 1-28 show configurations and features of various robotic device drive systems. Where possible, like numbers refer to similar elements to facilitate comprehension of the variety of implementations, uses, and configurations. Many of the figures are shown without the interventional devices installed for clarity and ease of explanation of the various mechanisms.

Controllers described herein may include one or more processors. Controllers may include the capability of multiple inputs and multiple outputs. Controllers described herein may receive one or more inputs (e.g., user activated control inputs, measurements from one or more sensors, etc.) and respond with one or more outputs (e.g., actuating pumps, actuating valves, translating/adjusting robotic arms, etc.). Control inputs may be local or remote and may be connected to the controller via wires or wirelessly. Examples of local control inputs include control inputs located on embodiments of robotic device drive systems, or near embodiments of robotic device drive systems (e.g., a control console within the operating room or within an adjacent room that the robotic device drive system is in). Examples of remote control inputs include control inputs performed from outside of the proximity of the robotic device drive system embodiment (e.g., outside the operating room, at another location, at a remote location, etc.). Remote control inputs may be performed by a user hundreds or even thousands of miles away from the robotic device drive system embodiment. Remote control inputs may be performed on a user interface and transmitted to the robotic device drive system embodiment by communication networks known in the art. Further, data, such as video signals, from robotic device drive system embodiments may be transmitted to the user interface and displayed for the user on the user interface.

Proximal and distal are used herein to describe directions with respect to embodiments of robotic device drive systems described herein. Proximal is defined herein as the direction away from a patient being treated by the robotic device drive system. Distal is defined herein as the direction towards the patient being treated by the robotic device drive system.

FIGS. 1-10 illustrate an embodiment of a robotic device drive system 100. The robotic device drive system 100 can include a rail 4, one or more robotic arms, and a fluidics system. For example, for certain procedures (e.g., neurovascular procedures), the robotic device drive system 100 may include five robotic arms 14, 16, 18, 20, 22 to control respective interventional devices. In other embodiments, the robotic drive system 100 can include one, two, three, four, six or any other suitable number of robotic arms.

FIG. 1 illustrates an embodiment of a robotic device drive system 100 in a contracted or stowed configuration. The contracted configuration may be suitable for storage or during inactive periods. The illustrated embodiment has five robotic arms 14, 16, 18, 20, 22 in a contracted or stowed state and segregated to one end (e.g., a proximal end) of the patient support device 2 (e.g., a patient support table). A first robotic arm 14, a second robotic arm 16, a third robotic arm 18, a fourth robotic arm 20, and a fifth robotic arm 22 are illustrated as functionally attached to a rail 4. The rail 4 can be attached to an elongate side 28 of the patient support device 2. The rail 4 may extend parallel to the elongate side 28 of the patient support device 2. As illustrated, the stowed arms 14, 16, 18, 20, 22 are opposite the patient 1, but could otherwise be stored anywhere along the length of patient support device 2.

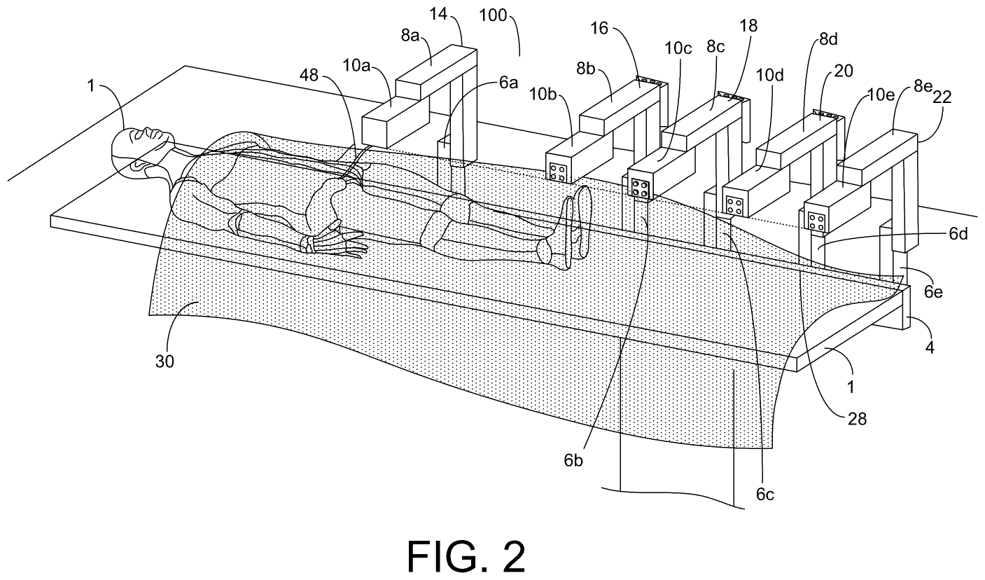

FIG. 2 illustrates an embodiment of a robotic device drive system 100 in an expanded configuration. The expanded configuration may also be referred to as a procedure configuration. In this configuration, the robotic arms 14, 16, 18, 20, 22 have moved or been translated from the storage configuration axially along the rail 4. The first robotic arm 14, which may be an introducer arm, can move from the storage configuration to a position adjacent or proximate to an access point vasculature (e.g., femoral artery, carotid artery, radial artery, etc.) of the patient 1. The first robotic arm 14 can translate along the rail 4 via a drive mechanism in communication with the rail 4. Examples of drive mechanisms that may move the first robotic arm 14 and/or any of the other robotic arms described herein include, but are not limited to: a rack and pinion system, a lead screw system, a ball bearing based system, or any other suitable mechanism known in the art.

The first robotic arm 14 can translate or move along the rail 4 via a first robotic arm bottom portion 6a and its associated drive mechanism. The bottom portion 6a of the first robotic arm 14 can be operatively coupled to a first robotic arm intermediate portion 8a, which can be attached to a first robotic arm head portion 10a. This configuration can move along the rail 4 and/or along the elongate side 28 of the patient support device 2 to a position predefined for the pending procedure.

The second robotic arm 16 can have a second robotic arm bottom portion 6b, a second robotic arm intermediate portion 8b, and a second robotic arm head portion 10b. The second robotic arm 16 can move along the rail 4, similar to the first robotic arm 14 as described above, to a position predefined for the pending procedure.

The third robotic arm 18 can have a third robotic arm bottom portion 6c, a third robotic arm intermediate portion 8c, and a third robotic arm head portion 10c. The third robotic arm 18 can move along the rail 4, similar to the first robotic arm 14 as described above, to a position predefined for the pending procedure.

The fourth robotic arm 20 can have a fourth robotic arm bottom portion 6d, a fourth robotic arm intermediate portion 8d, and a fourth robotic arm head portion 10d, The fourth robotic arm 20 can move along the rail 4, similar to the first robotic arm 14 as described above, to a position predefined for the pending procedure.

The fifth robotic arm 22 can have a fifth robotic arm bottom portion 6e, a fifth robotic arm intermediate portion 8e, and fifth robotic arm head portion 10e. The firth robotic arm 22 can move along the rail 4, similar to the first robotic arm 14 as described above, to a position predefined for the pending procedure. Although five arms are shown in a procedure configuration in FIG. 2, it should be appreciated that any subset of the arms may be stowed, such that not all arms are used for every procedure. Additionally, in some embodiments, one or more of the arms (e.g., a proximal most arm) may remain at the same position between the stowed configuration and the procedure configuration.

The first robotic arm 14, in some embodiments, may be used as an introducer arm. As an introducer arm, the first robotic arm 14 may provide for placement of a guide tube 48. The guide tube 48, can guide an interventional device (e.g., a guidewire or catheter) or an interventional device assembly (e.g., a concentrically oriented interventional device assembly) into the access point vasculature of the patient 1. The guide tube 48 may be rigid in some embodiments.

In some embodiments, the guide tube 48 can be configured to guide a first interventional device (e.g., a first concentric catheter) into the access point vasculature of the patient 1. The first catheter can be manipulated by the second robotic arm 16. In some cases, a second interventional device (e.g., a second smaller outer diameter catheter or a guidewire) is concentrically positioned inside the first catheter that is manipulated by the second robotic arm 16. The second interventional device can be can be manipulated by the third robotic arm 18. In some embodiments, when the second interventional device is a second catheter, a third interventional device (e.g., a third smaller outer diameter catheter or a guidewire) is concentrically placed inside the second catheter that is manipulated by the third robotic arm 18. The third interventional device can be manipulated by the fourth robotic arm 20. In some embodiments, a fourth interventional device (e.g., a fourth a smaller outer diameter catheter or a guidewire) is concentrically positioned inside the third catheter that is manipulated by the fourth robotic arm 20. The fourth interventional device can be manipulated by by the fifth robotic arm 22.

In certain embodiments, each more proximal concentric interventional device gains access to an adjacent distal device via a hemostasis valve or the like in the adjacent distal device's hub. The axial movement of each of the devices is accomplished via the translation of the corresponding arm along the rail 4.

Although five robotic arms 14, 16, 18, 20, 22 are illustrated, embodiments comprising more or less arms, for example one arm, two arms, three arms, four arms, or five arms, are further contemplated. For example, for a diagnostic procedure, robotic arm 14 and robotic arm 16 may be used. In further examples, for a peripheral vasculature procedure, robotic arm 14, robotic arm 16, and robotic arm 18 may be used. In still further example, for a coronary procedure, robotic arm 14, robotic arm 16, robotic arm 18, and optionally robotic arm 20 may be used. For example, for a neurovasculature procedure, robotic arm 14, robotic arm 16, robotic arm 18, robotic arm 20, and robotic arm 22 may be used.

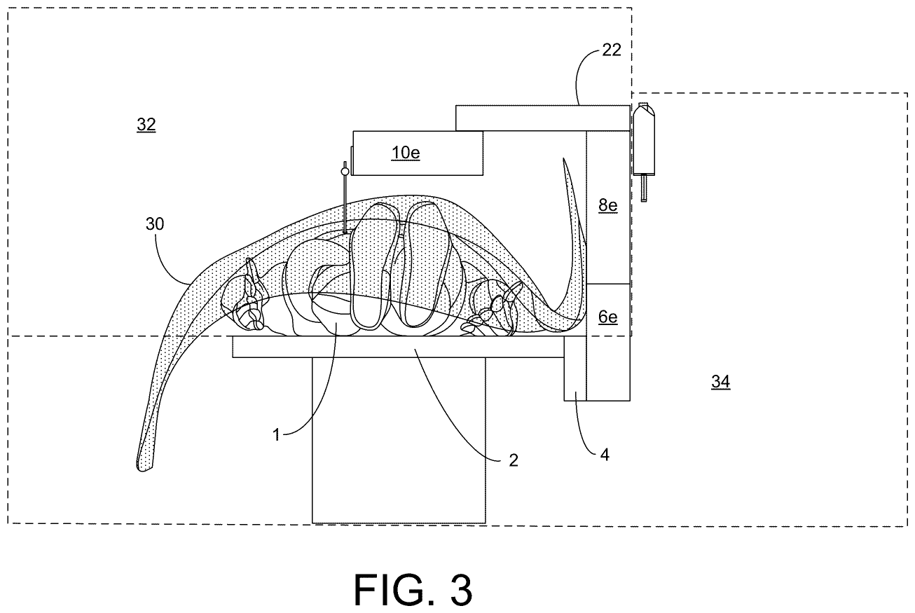

A patient draping boundary 30 and a separation of the sterile area 32 and a non-sterile area 34 are shown in FIG. 2 and FIG. 3. Additionally, the position and relationship of the robotic arm 22 with respect to sterile area 32 and non-sterile area 34 is shown in FIG. 3. The sterile arms 14, 16, 18, and 20 may be positioned in a similar relationship with respect to the sterile area 32 and non-sterile area 34.

In some embodiments, at least a portion of an arm can be draped and in a non-sterile area while the hub and associated interventional device above the patient is in sterile area. A sterile adapter between the hub and the arm may provide an interface between sterile area and non-sterile area.

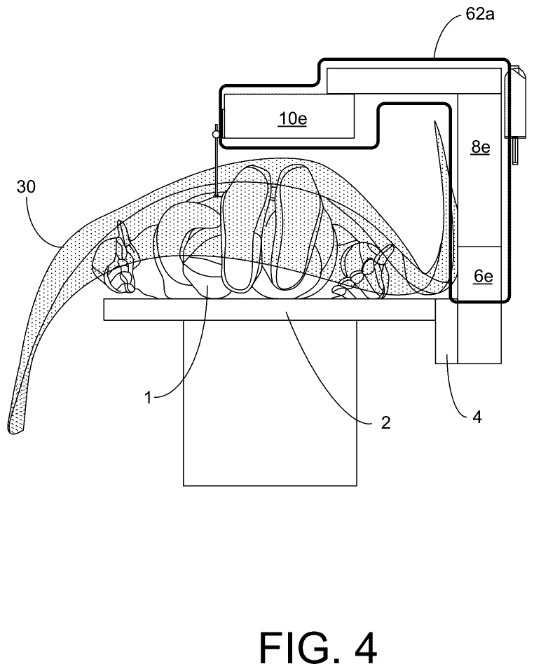

In an embodiment shown in FIG. 4, a drape 62a encapsulates arm 22 while not preventing translation of arm 22 along rail 4 attached to patient support device 2. It is contemplated that these draping solutions can be used on any of the robotic device drive systems described herein.

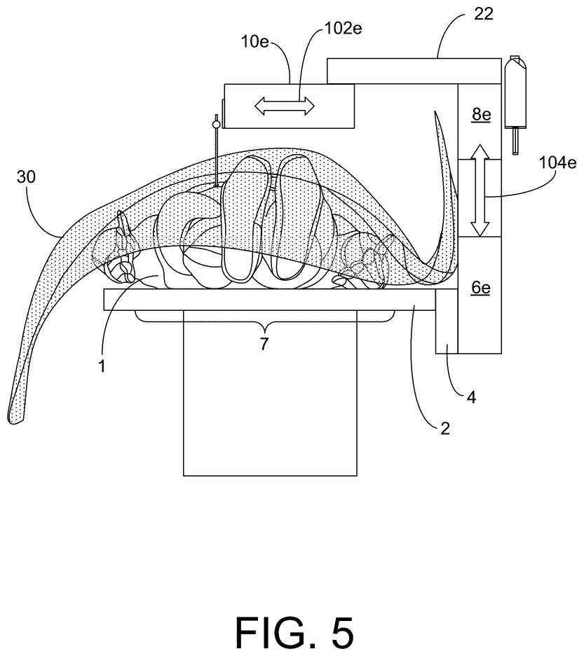

FIG. 5 depicts a proximal end 7 of the patient support device 2. FIG. 5 illustrates various translational capabilities a robotic arm of the system may have, shown as an example with respect to robotic arm 22. Any one or more of these translational capabilities of arm 22 may be shared amongst the other robotic arms 14, 16, 18, 20. The illustrated embodiment has three body components: the bottom portion 6c, intermediate portion 8e, and head portion 10c. The robotic arm 22 is vertically adjustable, shown via adjustment 104e, via movement of the intermediate portion 8e away from or towards the bottom portion 6e. The intermediate portion 8c and the bottom portion 6e are operatively coupled together. The intermediate portion 8c may be adjustable between a first height with respect to a lateral surface of the patient support device 2 and a second height with respect to the lateral surface of the patient support device 2. For example, the first height may be a storage height and the second height may be a procedure height. Although two positions are described, any number of intermediate positions are contemplated between the two heights. Alternatively, or additionally, a height or position of an intermediate arm 8e may change during the course of a procedure as the arm translates along the patient support device, for example to avoid a body portion of a patient or to maintain a device stack profile while suspending over at least a portion of the patient support device 2. A device stack profile is the longitudinal profile of one or more interventional devices (e.g., a guidewire within a catheter within another catheter). The longitudinal profile may include the heights, axial positions, lateral positions, and/or angles of the interventional devices along their lengths.

In addition, as shown in FIG. 5, the head portion 10e and the intermediate portion 8c can be operatively coupled together. This coupling can allow for horizontal adjustment, shown via adjustment 102e. Although horizontal adjustments 102e and vertical 104e adjustments are shown, embodiments with no adjustable features, horizontal adjustment, vertical adjustment, or both horizontal and vertical adjustment are contemplated. The adjustment mechanisms may include, but are not limited to, hydraulic pistons, pneumatic pistons, linear actuators, electric step motors, and the like. Position tracking of arm portions may include optical encoders, potentiometers, and the like.

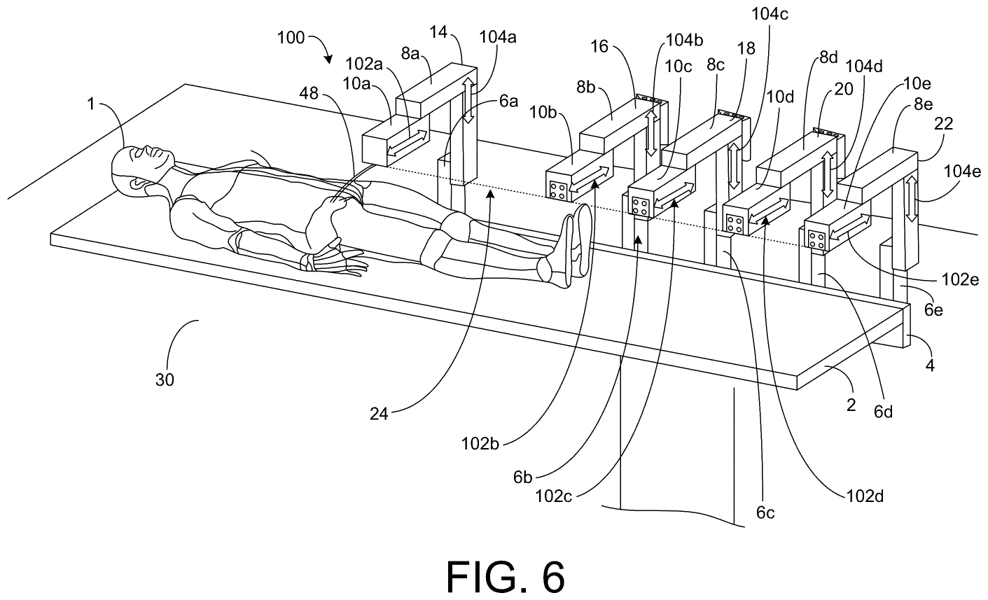

FIG. 6 illustrates an embodiment of a robotic device drive system 100 in the expanded position showing horizontal adjustment features 102a, 102b, 102c, 102d, 102e and vertical adjustment features 104a, 104b, 104c, 104d, 104e of each arm. As described above with respect to FIG. 5, each arm 14, 16, 18, 20, 22 may be configured to adjust horizontally and/or vertically to manipulate the device stack profile 24.

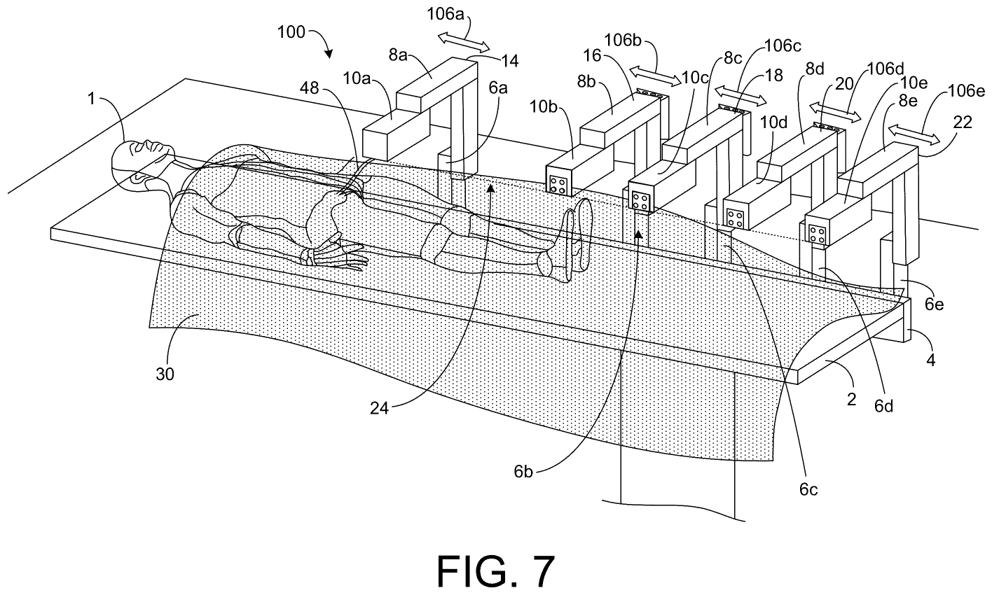

FIG. 7 illustrates an embodiment of a robotic device drive system 100 in an expanded configuration. FIG. 7 depicts the axial adjustment, shown via arrows 106a, 106b, 106c, 106d, 106e, of the respective arms 14, 16, 18, 20, 22. Axial adjustments as shown by arrows 106b, 106c, 106d, 106e can provide retraction and advancement of the corresponding devices of the arms 16, 18, 20, 22. In the illustrated embodiment, the arrow 106a shows axial adjustment of the first robotic arm 14 for the positioning of the guide tube 48 for the pending procedure.

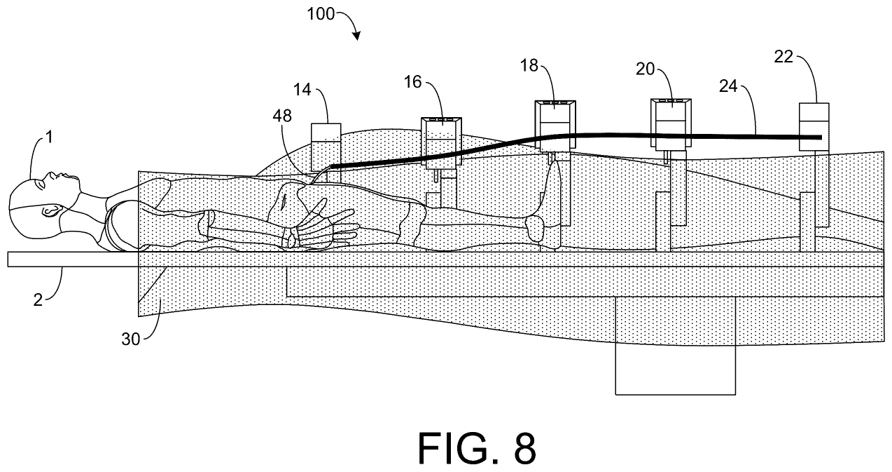

FIG. 8 illustrates an embodiment of a robotic device drive system 100 in an expanded configuration and a side view of the device profile 24. As described above the vertical adjustments of the arms 14, 16, 18, 20, 22 allow for the adjustment of the device stack profile 24. For example, as in FIG. 8, if the feet of the patient 1 obstruct the device stack profile 24, the device stack profile 24 can be adjusted to a position above the obstruction. For example, one or more of the arms (e.g., arms 18, 20, and 22) may be raised so that the interventional devices within the device stack do not contact the feet as the interventional devices are moved relative to the patient. The device stack profile 24 can be created with attention paid to critical radius parameters of each device. In other words, catheter type devices can have bend radius limits. If these bend radius limits are exceeded, kinking and buckling can become reasonable concerns. It is further contemplated that embodiments with control systems may have control protocols that do not allow operations outside of these parameters.

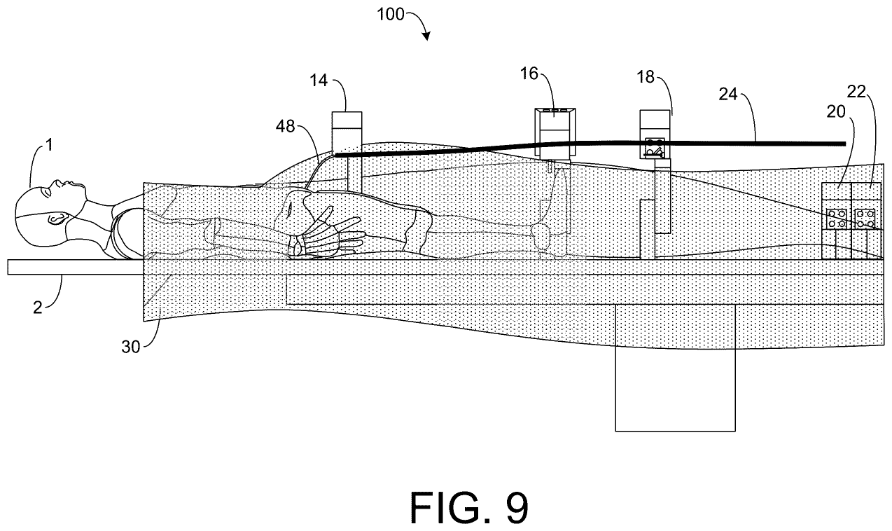

FIG. 9 illustrates a partially expanded configuration of a robotic device drive system. In this configuration, the embodied system can be used for procedures not requiring all robotic arms 14, 16, 18, 20, 22. The configuration of FIG. 9 may also be a stage configuration showing the robotic device drive system at a particular stage of a procedure at which point all arms are not in use, but may be transitioned to an expanded configuration during another stage of the procedure. In this illustrated embodiment, the first robotic arm 14, the second robotic arm 16, and the third robotic arm 18 are in use or deployed, while the fourth robotic arm 20 and fifth robotic arm 22 are in the contracted or stowed configuration. For example, this configuration could be used in a procedure in which the first robotic arm 14 acts as an introducer by positioning the guide tube 48 in proximity to the access point, the second robotic arm 16 is used for manipulation of a catheter and the third robotic arm 18 is used for manipulation of a guidewire within the catheter. The arms, or a subset thereof, may be configured to move in tandem, in a coordinated fashion, on a one-by-one basis, or the like, depending on user input from a set of controls or based on preconfigured settings for a procedure.

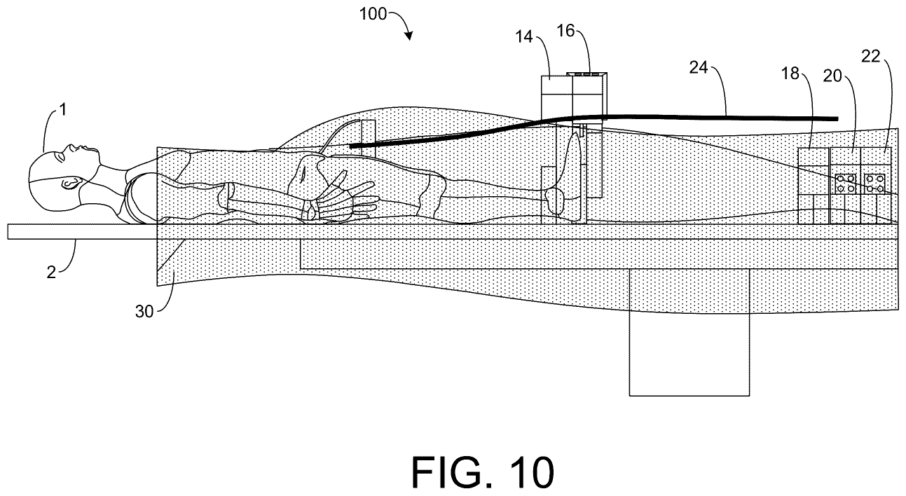

FIG. 10 illustrates an embodiment of a robotic device drive system 100 configured with the first robotic arm 14 and second robotic arm 16 deployed. An example use of this configuration is as an aid during manual catheter stack manipulation.

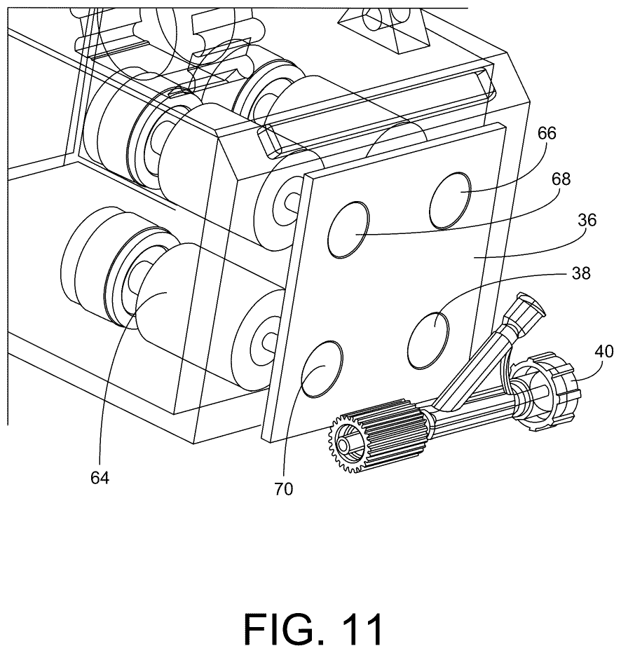

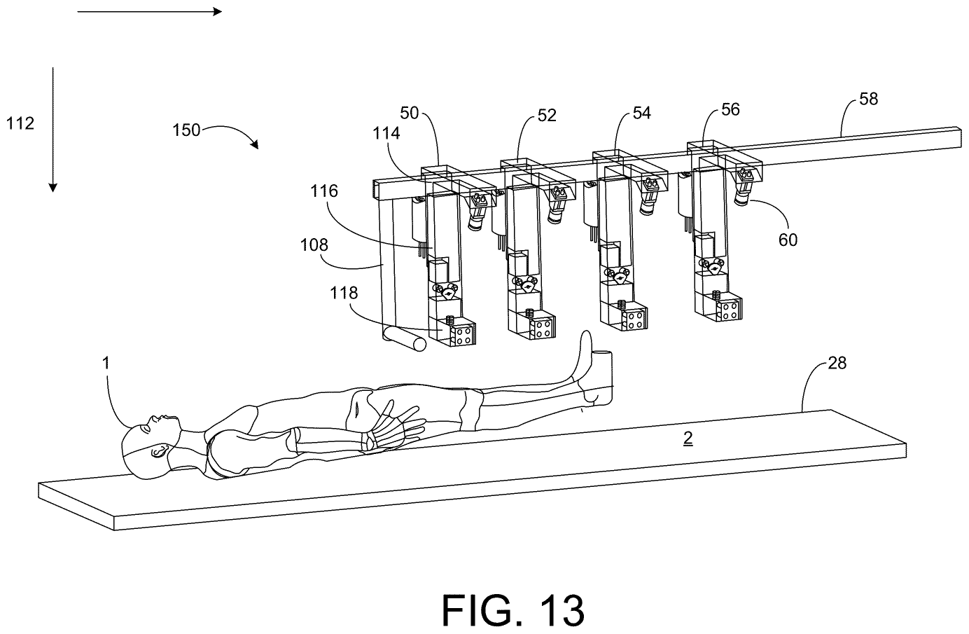

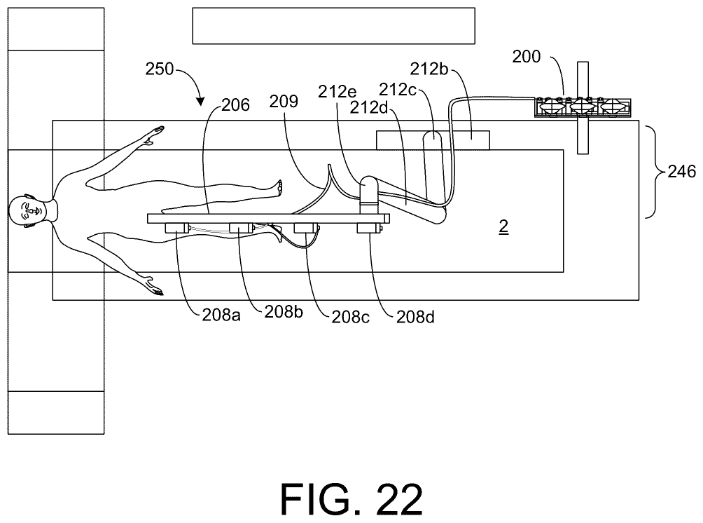

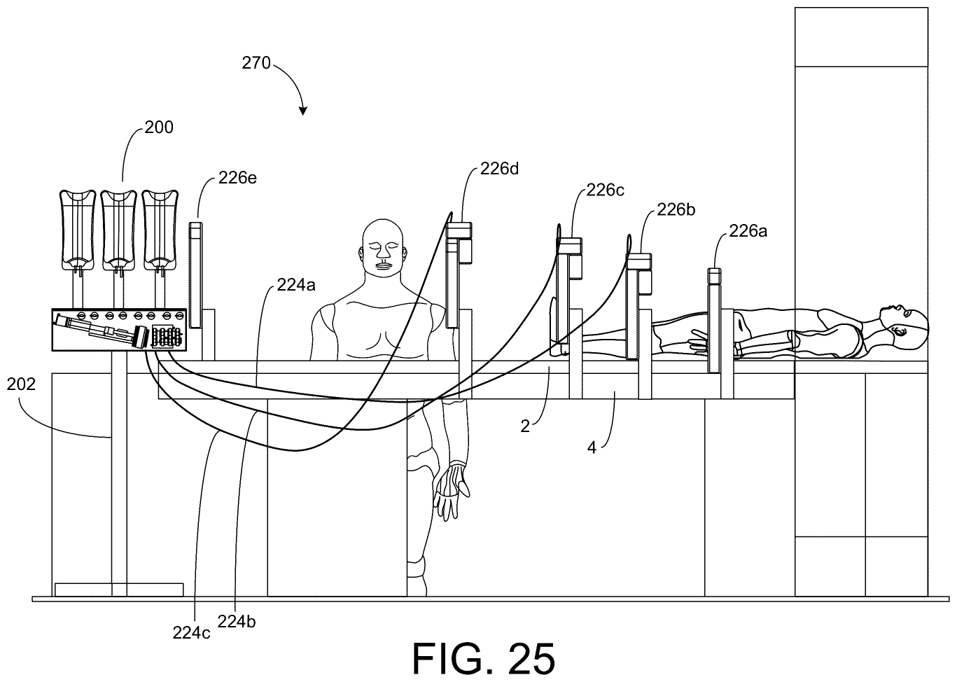

FIG. 11 illustrates an interface that can be used with any of the robotic arms 14, 16, 18, 20, 22 of FIGS. 1, 2, and 6-10, the robotic arms 50, 52, 54, 56 of FIG. 13, the hubs 208a, 208b, 208c, 208d of FIGS. 22-24, or the robotic arms 226a, 226b, 226c, 226d of FIG. 25. Various mechanical outputs are illustrated. The outputs may be provided (e.g., in response to one or more signals from a control system) to one or more drive mechanisms coupled to an interventional device, a hemostasis valve, and/or components (e.g., or a hub associated with the interventional device). The one or more drive mechanisms may be positioned within a hub associated with the interventional device or otherwise in communication with the hub associated with the interventional device, the interventional device, or the hemostasis valve. In some embodiments, the hub associated with an interventional device may be part of or coupled to a robotic arm head portion.

The mechanical outputs may be used during different portions of a procedure or for different procedures. In this embodiment, an arm comprises four control outputs (e.g., that are transmitted through the interface to the hub), although any number and arrangement of control outputs are contemplated herein. One output may be an actuator 38 of hemostasis valve 40. The hemostasis valve actuator 38, being powered by a drive system housed within any one of the robotic arms described herein, is capable of actuating the hemostasis valve 40 in the catheter hub (not shown). The hemostasis valve 40 may be manipulated between an open configuration to allow for removal of a device or introduction of an additional device (e.g., a microcatheter or guidewire) into the stack; an intermediate configuration for example for low pressure contrast injections; or closed configuration to isolate internal working pressures and/or to allow high pressure contrast injections, for example. Although three positions of the hemostasis valve are described, any number of positions or configurations are contemplated. Mechanisms for hemostasis valve 40 actuation may be similar to those described in U.S. patent application Ser. No. 17/879,614, filed Aug. 2, 2022, which is herein incorporated by reference in its entirety. Another output may be a roll actuator 70 for rotational control of an interventional device being manipulated by the hub (not shown), which translates into rotation control of the interventional device (e.g., catheters, guidewires, and etc.) coupled to the respective hub. The drive mechanism for the roll actuator 70 may be housed within any one of the robotic arms described herein and have a coupling interface on the hub side of the disposable sterile adapter 36. Mechanisms for roll actuation may be include a magnetic gear train, a mechanical gear train, and the like. A further output may be an articulating interventional device mechanism 66. Articulation mechanisms 66 for articulating an interventional device may include pullwire mechanisms, manipulation of concentrically disposed tubes that are at least partially bonded to one another, or other mechanisms known in the art. Another output may be an anti-buckling assist 68 with an associated drive mechanism housed within any one of robotic arms or hubs described herein. The deployable assembly of the anti-buckling assist 68 may be mounted in, onto, or integrated with the hub (not shown) of the disposable sterile adapter 36. For example, the anti-buckling assist 68 may be a telescoping tube assembly, a scissoring assembly, a spring-based assembly, a reel with a split tube, or the like. The split tube, of stiffer construction than the catheter for intended sleeving, can be operationally slid over a device (e.g., a catheter) for assistance against buckling. Furthermore, during advancement and retraction of the assisted device, the control system may control the advancement and retraction of the split tube via the reel and associated drive mechanism.

Turning now to various fluidic system configurations that are integrated in various ways with any of the robotic device drive systems described elsewhere herein.

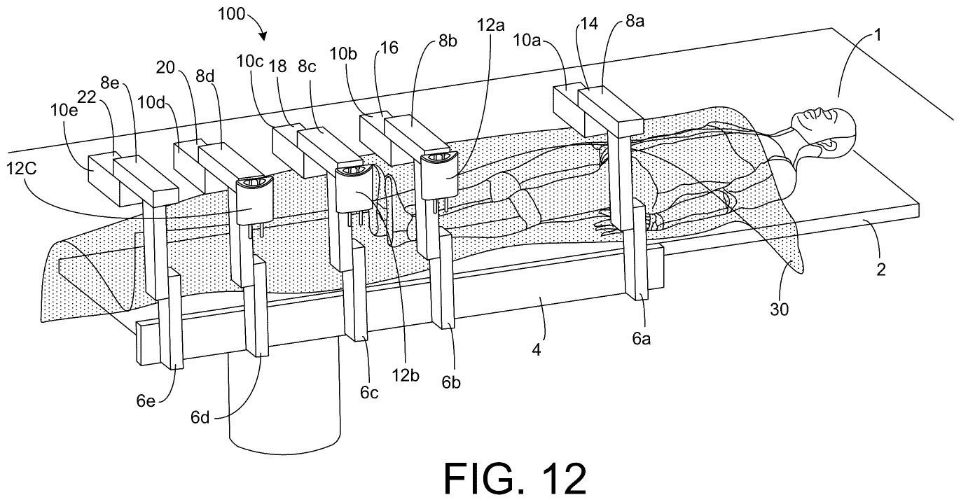

FIG. 12 illustrates an embodiment with fluid reservoirs or manifolds 12a, 12b, 12c attached to respective robotic arms 16, 18, 20. These fluid reservoirs or manifolds 12a, 12b, 12c can store fluids, such as saline, contrast, pharmaceuticals, or any other fluid, and/or control the release of fluids, within proximity to its respective catheter hub. Proximal storage and/or control of various fluids reduces the lengths of associated tubing. Shorter tubing lengths reduce bubble propagation issues, add to the overall organization of the system, prevent tubes from being pinched during translation of an arm, and the like. Additionally, these fluid reservoirs or manifolds 12a, 12b, 12c can be coupled to the arms via a load sensing device for the supervision of fluid levels by the system or user. Alternatively, a fluid reservoir may include a fluid sensor, such that when the fluid contained in the reservoir reaches a predefined level, an indicator is activated. Implementations and features of various fluidics configurations are shown and described in U.S. patent application Ser. No. 17/879,614, filed Aug. 2, 2022, which is herein incorporated by reference in its entirety. Although described as being attached to robotic arms 16, 18, 20, fluid reservoirs or manifolds, such as 12a, 12b, 12c, may be attached to any robotic arm described herein, for example, robotic arms 50, 52, 54, 56 of FIG. 13, or robotic arms 226a, 226b, 226c, 226d of FIG. 25.

FIG. 13 illustrates an embodiment of a robotic device drive system 150. The robotic device drive system 150 can include a rail 58, one or more robotic arms, and a fluidics system. For example, for certain procedures (e.g., neurovascular procedures), the robotic device drive system 150 may include robotic arms 50, 52, 54, 56 that translate along an overhead rail 58.

The overhead rail 58 can be parallel to the elongate side 28 of the patient support device 2. The overhead robotic arms 50, 52, 54, 56 may move along the overhead rail 58. While not shown in FIG. 13, in some embodiments, the rail 58 is supported by a column (or more than one column), running perpendicular to the rail 58, and supporting the rail 58 from the ground. In some embodiments, the rail 58 is supported by the procedure room ceiling (e.g., a beam or truss in the ceiling) by one or more vertical supports. In some embodiments, a handle 108 may be coupled to the rail 58 for adjusting the position of the rail 58.

The robotic arms 50, 52, 54, 56 can move along the rail 58 to advance and retract interventional devices that are coupled to each of the respective arms. Each of the robotic arms 50, 52, 54, 56 may have a base portion 114 with a respective translation mechanism for movement along the rail 58. In addition, the use of an intermediate portion 116, operatively coupled to the base portion 114 via an adjustment drive mechanism, may be used for adjustment along a vertical 112 axis. A third translational degree of freedom may be performed by the manipulation of a head portion 118. The head portion 118, operatively coupled to the intermediate portion 116 via a corresponding adjustment mechanism, may accomplish adjustment in the translational axis perpendicular to both the horizontal 110 and vertical 112 axes. Although illustrated with four robotic arms 50, 52, 54, 56, embodiments with less or more arms are further contemplated. Further, as described elsewhere herein, although three portions of each arm are shown, an arm of the system may include one portion, two portions, or a plurality of portions. Further, although adjustment of the head portion relative to the intermediate portion is shown and adjustment of the intermediate portion relative to the base portion is shown, neither of these adjustments may be implemented, one of the adjustments may be implemented, or both of the adjustments may be implemented.

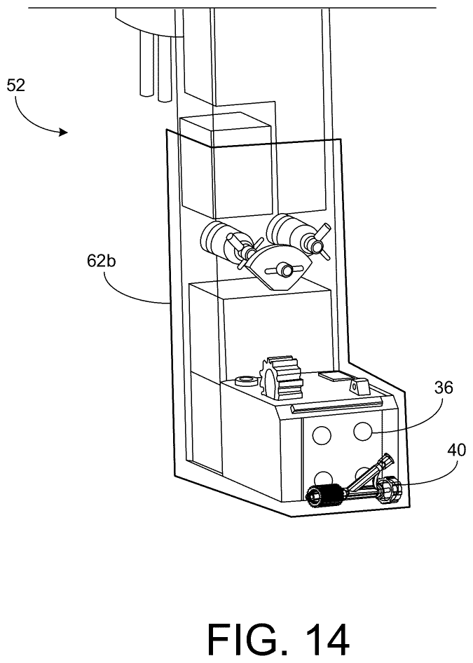

FIG. 14 illustrates an example of a drape 62b. The drape 62b can be placed over the arm 52 and aid in demarcating sterile and non-sterile fields. The example drape 62b may be used on any of the robotic arms 50, 52, 54, 56.

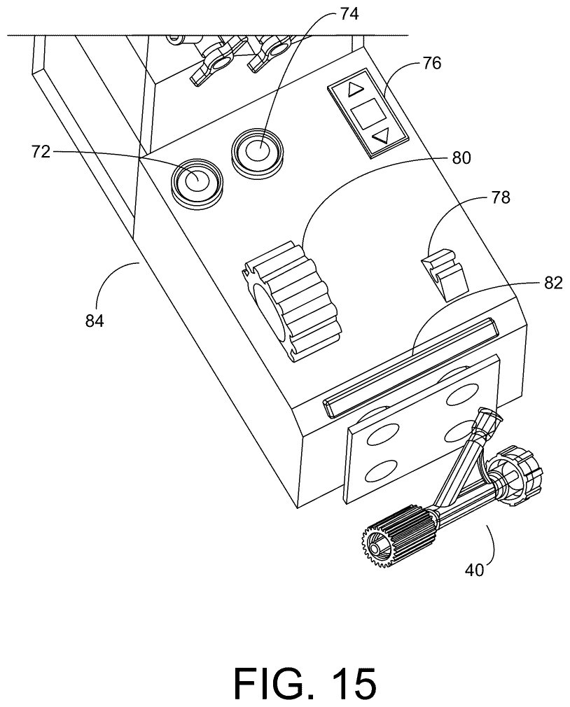

FIG. 15 illustrates an embodiment of a local system user interface 84 of a robotic device drive system. The illustrated embodiment depicts the user interface 84 as positioned on one of the robotic arms 50, 52, 54, 56 of the system of FIG. 13. The user interface 84 may be placed anywhere suitable on one or more of the robotic arms 50, 52, 54, 56. For example, user interface 84 may be positioned on a head portion, intermediate portion, or a base portion of an arm. Additionally, the user interface 84 may be positioned on, and used with, any robotic arm described herein (e.g., robotic arms 14, 16, 18, 20, 22 of FIGS. 1, 2, and 6-10, hubs 208a, 208b, 208c, 208d of FIG. 2, or robotic arms 226a, 226b, 226c, 226d of FIG. 25). Alternatively, user interface 84 may be positioned on a remote device, for example a controller, fob, computing device (via an application running on the computing device), and the like, such that there is a wired connection (e.g., via databus) or wireless connection (e.g., via an antenna or coil) between the robotic system and the remote user interface.

An input depicted in the user interface 84 is an enablement input 82. This enablement input 82 may be a momentary depression button or other input type acting as a prerequisite for any other inputs of the user interface 84. Acting as a safety against accidental manipulation of other control inputs, the enablement input 82 may reduce unintended control outputs.

Another contemplated input is one or more device actuation input controls 80. When actuated, a device actuation input control 80 may cause a drive mechanism to axially translate and/or rotate an interventional device coupled to a respective hub of the corresponding arm. Although a wheel mechanism is shown, one of skill in the art will appreciate that a joystick, a series of buttons, a touchpad, or the like may function as a device actuation input control. As described elsewhere herein, additionally, or alternatively, a device may be axially translated based on arm movement relative to the patient support device.

One or more fluid injection controls 72 may be used for the injection control of appropriate fluids, for example contrast or saline. An injection control 72 may comprise a depressible button or other control element such that, when activated, causes a valve in the line, hub, or hemostasis valve to be opened to release the desired fluid into a lumen of an interventional device coupled to the line, hub, or hemostasis valve.

An aspiration control 74 may also be within the user interface 84. The aspiration control 74 may comprise a depressible button or other control element, such that when activated, causes opening of a valve in a vacuum line and/or activation of vacuum pump so that suction is applied through a lumen of an interventional device coupled to the line, hub, or hemostasis valve.

The user interface 84 may optionally include a bubble detection and/or removal system comprising one or more bubble sensors 78. A bubble sensor 78, installed on or in proximity of a corresponding fluid line, can detect the presence of a bubble in the line. When a bubble is detected, the control system may alert the user or enter an autonomous bubble removal protocol for the respective fluid line. Use of a bubble sensor 78 may greatly reduce the introduction of a bubble into a patient's vasculature. Any of the embodiments described herein may benefit from a bubble removal system and such has been contemplated. For example, a bubble removal system can be automatically activated upon detection of in line bubbles. A controller may be configured to activate a valve positioned in the flow path downstream of the bubble detector, upon the detection of bubbles. The valve can divert a column of fluid containing the detected bubble out of the flow path leading to the patient and instead into a bypass flow path or reservoir. Once bubbles are no longer detected in the flow path and after the volume of fluid in the flow path between the detector and the valve has passed through the valve, the valve may be activated to reconnect the source of fluid with the patient through the flow path. In some embodiments, the flow path may include any number of bubble filters and/or traps to remove bubbles from the flow path.

The user interface may optionally further include one or more drip rate controls 76 for selecting flow rates of appropriate fluids (e.g., saline). A drip rate control 76 may be used to control the rate fluid is introduced through the corresponding line into a lumen of an interventional device that is fluidly connected to the line. A drip rate control 76 may include a feedback display illustrating the selected fluid rate. A positive display rate may be for normal fluid rates and a negative display rate may be for negative pressure. The various controls discussed herein as well as any other controls may be embodied in a local user interface 84 as discussed above or located remotely.

The uses and actions described herein may be controlled manually, through use of a user interface, may be performed autonomously via the control system (e.g. programmable logic), or a combination of both. Robotic arm or hub movements described herein may be performed independently of other arms or hubs, or in an orchestrated manner with other arms or hubs (e.g., master/slave protocols). Embodiments exploiting programmable logic may incorporate master/slave protocols for the movement of the arms. For example, if any one of the arms have a change in position of their corresponding head portion, one or more of the other arms, not under active user control, may mimic the movements either proportionally or to scale. This method may be used in step elimination, further simplifying use, or as avoidance of unwanted scenarios. Unwanted scenarios may include device stack profiles with steep changes that add to the possibility of kinking or buckling.

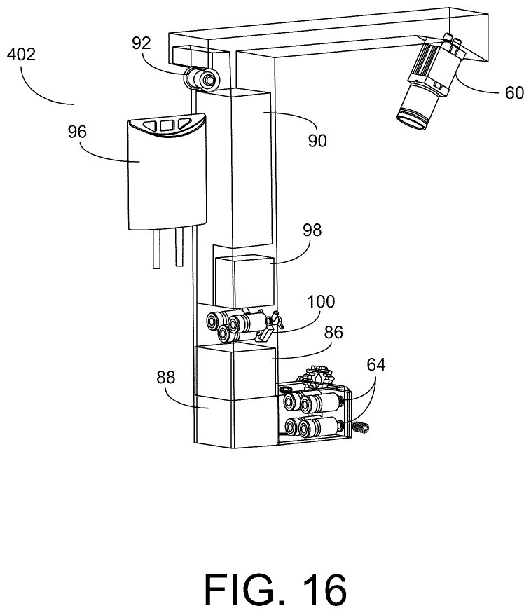

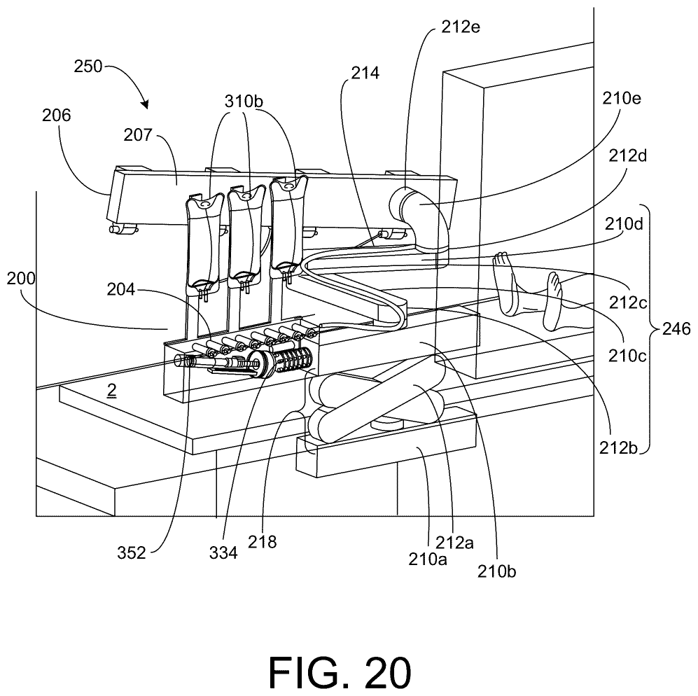

FIG. 16 illustrates an embodiment of a robotic arm. The features of the robotic arm of FIG. 16 may be implemented in any of the embodiments described elsewhere herein, including table mounted embodiments as well as ceiling or scaffolding mounted embodiments. Additionally, the illustrated robotic arm of FIG. 16 may be representative of any one of the hubs 208a, 208b, 208c, 208d of the embodiment of FIGS. 20-24. In FIG. 16, the robotic arm is shown as semi-transparent to illustrate internal systems and features. The drive mechanisms 64 for the mechanical outputs at the hub interface are illustrated in FIG. 16, including anti-buckling assist, articulating insert catheter, device roll, and hemostasis valve controls described in connection with FIG. 11. These mechanisms can be housed inside the body (e.g., head portion, intermediate portion, bottom portion) of the arm 402 and operatively coupled to the hub for interventional device manipulation.

Additionally, or alternatively, a fluidics system (source system) and/or aspiration system (sink system) may be integrated into a body of an arm, such as any of the arms described elsewhere herein. For example, a vacuum reservoir 88 (for expelled fluid from an interventional device) 88 and vacuum pump 86 may be housed within the arm 402. The vacuum reservoir 88 and vacuum pump 86 may be fluidly connected (e.g., via tubing running at least partially in the body of the arm) to the hub and thus fluidly connected (e.g., via a hemostasis valve) to a lumen of an interventional device. Alternatively, one or both of the vacuum reservoir 88 and pump 86 may be located outside of the body of the arm, for example for easy emptying of the reservoir or visualization of fluid or particulate coming through the line or reservoir. Aspiration may be controlled by aspiration control 74 described above.

Further contemplated, a peristaltic saline pump 98 may be used to supply appropriate fluid to the corresponding hub and associated interventional device. The pump and fluid rate may be controlled, at least in part, by drip rate sensor 76. Some embodiments may include an external fluid reservoir 96 for storage of procedural fluids. The external fluid reservoir 96 may include a load sensor or other weight sensor to monitor fluid use during a procedure. The saline pump 98 may be connected to an external fluid reservoir 96 to supply fluid (i.e., administer fluid).

Further, a contrast injection system 90 (e.g., comprising a contrast fluid reservoir and charge pump) may be housed within the arm 402 for the supply of contrast fluid to a corresponding hub connected to an interventional device. The contrast injection system may be fluidly connected (e.g., via tubing at least partially installed within a body of the arm) to a hub of the system and thus fluidly connected (e.g., via a hemostasis valve) to a lumen of a catheter.

Further, any of the fluid systems (e.g., contrast, saline, etc.) described herein may be tilted at an angle away from an output fluid line, such that any bubbles that may be in the fluid system have an increased likelihood of remaining in the reservoir near a top of the fluid, away from the output line.

Optionally, any of the systems described herein may include a camera 60 that may be mounted in a suitable position for observation of a procedure. For example, a camera may be integrated into a portion of the arm. The use of one or more cameras can provide visualization when operations are performed remotely. Alternatively, one or more cameras may be installed in a procedure room, separate from the robotic device drive system.

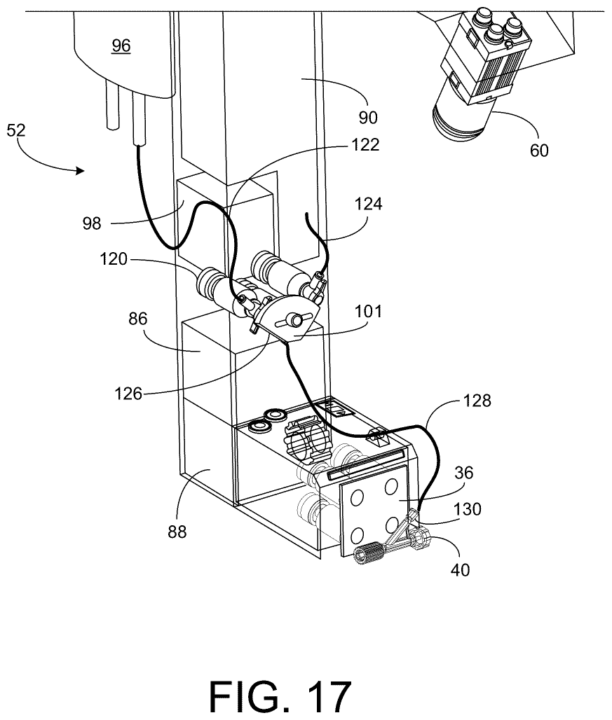

FIG. 17 depicts an embodiment of an arm of a robotic device drive system showing various tubing that couples a fluidic system to a corresponding hub and hemostasis valve 40. A four-port manifold 101 is depicted in the embodiment of FIG. 17. The manifold 101 is depicted as having a plurality of input lines that are integrated into an output line fluidly connected to the hemostasis valve of the hub. The plurality of input lines into the manifold 101 depicted are: an aspiration line 126 fluidly connected to an aspiration pump 86 and reservoir (sink), a saline line 122 fluidly connected to a saline pump 98 fed by an external fluid reservoir 96 (source), and a contrast line 124 fluidly connected to a contrast injection system 90 (source). The plurality of input lines is in fluid communication with an output line of manifold 101 connected to output line 128, which ultimately fluidly connects the manifold 101 to the hemostasis valve 40 in the hub. The manifold 101 depicted may have three valves for the introduction or removal of fluid communication of the respective input lines 122, 124, 126 to the output line 128. Other embodiments may include a manifold 101 with a valve stem selectively controlling the isolation and fluid communication of the input lines 122, 124, 126 to the output line 128. For example, one position of the valve stem may provide fluid communication with the saline line 122 to the output line 128, and simultaneously isolate (i.e., block) both the contrast line 124 and the aspiration line 126. A second position of the valve stem may provide fluid communication with the aspiration line 126 to the output line 128, and simultaneously isolate both the saline line 122 and the contrast line 124. A third position of the valve stem may provide fluid communication with the aspiration line 126 to the output line 128, and simultaneously isolate both the contrast line 124 and the saline line 122. With respect to the manifold 101 lines or line (e.g., output, input, etc.) may be synonymous with ports or port, respectively. The outlet line 128 may couple to a fluid port 130 of the hemostasis valve 40.

Additionally depicted in this embodiment are drive mechanisms 120 for the one or more valves in the manifold 101. For example, the drive mechanisms may be electric motors with appropriate gear trains operatively coupled to the valves of the manifold 101 to manipulate the valves between open or closed states. With the electric drive mechanisms 120, the control system of certain embodiments may autonomously actuate valves or actuate after certain conditions and/or inputs are realized. The drive mechanisms 120 of certain embodiments may have gear ratios appropriate (e.g., low ratios) to allow a user to back drive the motor when manual manipulation of the one or more valves is desired. The one or more valves of these embodiments have both drive mechanisms 120 and manual levers. Some embodiments may use a disposable, single-use manifold 101 attached to the arm 52 with a sterile adapter and under draping. It is further contemplated, that the output line 128 may pass through the draping to connect to the hemostasis valve 40 in the hub.

The embodiments of fluidic systems described above may drastically improve the efficiency of procedures and reduce inherent risks. The proximity of one or more fluid reservoirs, one or more fluid pumps (e.g., peristaltic pump), and/or one or more vacuum pumps may greatly reduce the lengths of fluid lines required. Reducing fluid line lengths can reduce the probability of bubble propagation, and thus, improves the patient safety. Additionally, the reduction of fluid lines can improve the organization of the system. With self-contained fluidic systems within the arms, many external fluid lines are eliminated. Additional details of fluidic systems and related features may be found in U.S. patent application Ser. No. 17/879,614, entitled Multi Catheter System with Integrated Fluidics Management, filed Aug. 2, 2022, which is herein incorporated by reference in its entirety.

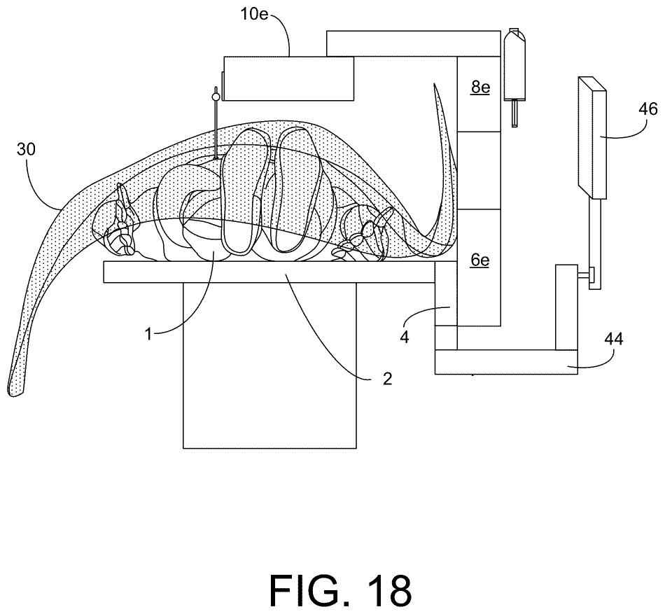

FIG. 18 illustrates an embodiment of a robotic device drive system with an accessory rail 44 for optional attachment of one or more supplementary devices 46. The accessory rail may be coupled to patient support device 2, for example below a rail 4 for the one or more robotic arms or along an opposite side, or an end of the patient support device 2. The accessory rail 44 may be used with any robotic device drive system described herein.

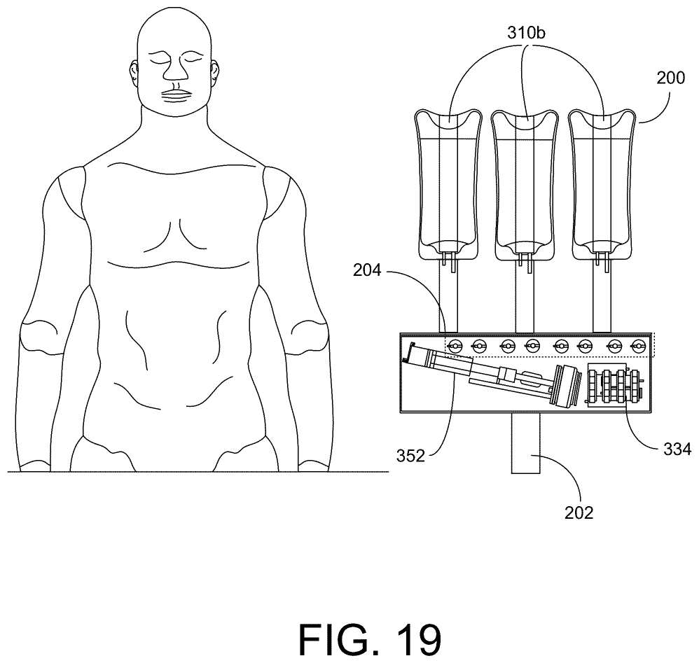

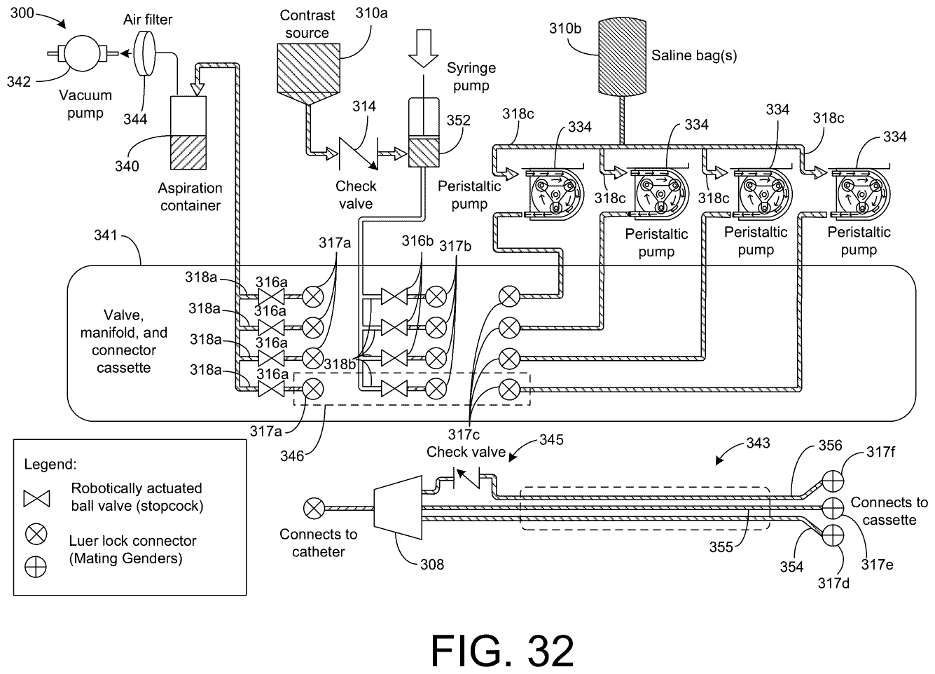

Illustrated in FIG. 19, some embodiments described herein may include a standalone fluidics system 200. The standalone fluidics system 200 may include one or more external fluid reservoirs 310b, a contrast injection pump 352, one or more peristaltic pumps 334, and/or a valve manifold 204. The valve manifold 204 may include a plurality of automated valves. Further, the fluidics system 200 may include a vacuum pump 342 and an aspiration canister 340 (illustrated in FIG. 32). The standalone fluidics system 200 may include a base 202 for supporting the fluidics system 200 upon the floor or ground. Alternatively, the fluidics system 200 may be coupled to a patient support device 2 (i.e., an operating table) illustrated in FIG. 20. A standalone fluidics system 200 may be used in place, or in conjunction with, fluidics systems integrated within the robotic arms or hubs of embodiments described herein.

As illustrated in FIG. 19, the valve manifold 204 may include a plurality of automated valves. The valve manifold 204 may include the capability of coupling a plurality of fluid sources and/or sinks to a plurality of interventional devices. A plurality of fluid lines may extend between a source or sink and the valve manifold 204 for coupling to different interventional devices. The valve manifold 204 may include some, or all, of the capabilities of the cassette 341 described for FIG. 32. For example, the actuation of automated valves within the valve manifold 204 may direct fluid flow to or remove fluid flow from one or more interventional devices. Based on local or remote control inputs, the controller may actuate one or more automated valves to direct or remove fluid flow, such as, contrast or saline, to one or more interventional devices. The contrast fluid directed to an interventional device may be pressurized by the contrast injection pump 352. The contrast injection pump 352 may include a linear actuated syringe pump. The saline directed to an interventional device may be pressurized by one or more peristaltic pumps 334. Further, the controller may direct or remove aspiration to one or more interventional devices.

FIGS. 20-24 illustrate an embodiment of a robotic device drive system 250. The robotic device drive system 250 can include a drive table or procedure rail 206, one or more hubs 208a-d, and a fluidics system 200. For example, for certain procedures (e.g., neurovascular procedures), the robotic device drive system 250 may include a procedure rail 206, a rail adjustment assembly 246, a fluidics system 200, and a patient support device 2. The procedure rail 206 may be operatively coupled to hubs 208a, 208b, 208c, 208d (illustrated in FIGS. 22-24) allowing the hubs to translate axially along the procedure rail 206. The rail adjustment assembly 246 may include a first rigid support 210a, a second rigid support 210b, a third rigid support 210c, a fourth rigid support 210d, and a fifth rigid support 210e. Further, the rail adjustment assembly 246 may include adjustment mechanisms, including, a first mechanism 212a, a second mechanism 212b, a third mechanism 212c, a fourth mechanism 212d, and a fifth mechanism 212c.

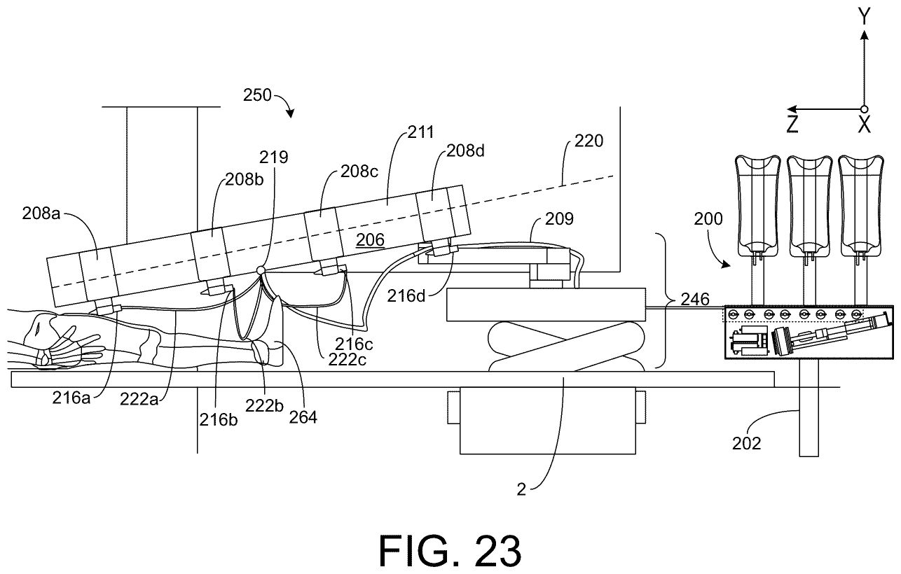

The first mechanism 212a can operatively couple the first rigid support 210a and the second rigid support 210b. The first mechanism 212a (e.g., a scissor mechanism) may include a mechanism for adjusting the distance 218 between the first rigid support 210a and the second rigid support 210b. Adjusting the distance 218 can adjust the height of the procedure rail 206 with respect to the y-axis (e.g., as shown in FIG. 23).

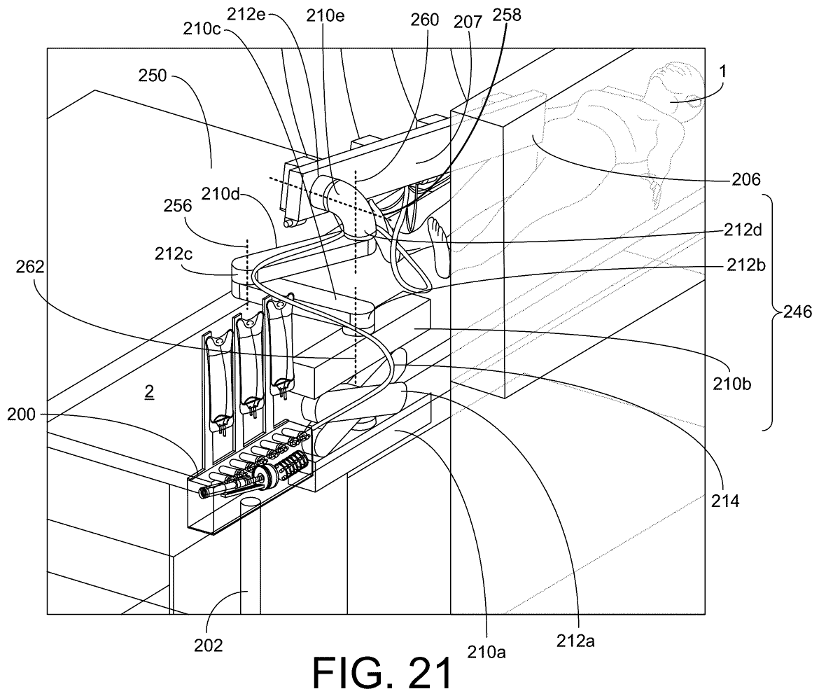

The second mechanism 212b may operatively couple the second rigid support 210b to the third rigid support 210c. The second mechanism 212b (e.g., a revolute joint) may allow for rotation of the third rigid support 210c about the axis 262 (illustrated in FIG. 21) and with respect to the second rigid support 210b.

The third mechanism 212c may operatively couple the third rigid support 210c to the fourth rigid support 210d. The third mechanism 212c (e.g., a revolute joint) may allow for rotation of the fourth rigid support 210d about the axis 256 (illustrated in FIG. 21) and with respect to the third rigid support 210c.

The fourth mechanism 212d may operatively couple the fourth rigid support 210d to the fifth rigid support 210e. The fourth mechanism 212d (e.g., a revolute joint) may allow for rotation of the fifth rigid support 210e about the axis 260 (illustrated in FIG. 21) and with respect to the fourth rigid support 210d.

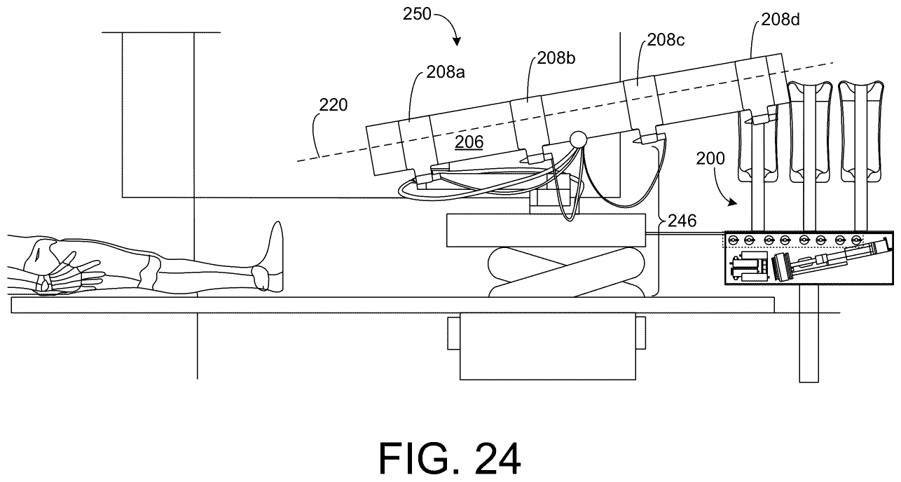

The fifth mechanism 212e may operatively couple the fifth rigid support 210e to the procedure rail 206. The fifth mechanism 212e (e.g., a revolute joint) may allow for rotation of the procedure rail 206 about the axis 258 (also shown in FIG. 21) and with respect to the fifth rigid support 210c. The fifth mechanism 212e (e.g., functioning similar to a prismatic joint) may also allow the procedure rail 206 to translate along the rail longitudinal axis 220 (illustrated in FIGS. 23 and 24) with respect to the fifth rigid support 210e. The translation of the procedure rail 206 along the rail longitudinal axis 220 (illustrated in FIGS. 23 and 24) may be facilitated by a groove or rack operatively coupled to the fifth mechanism 212c.

The rail adjustment assembly 246 may support at least a portion of the weight of the procedure rail 206 and rail components (e.g., hubs 208a, 208b, 208c, 208d, etc.). The rail adjustment assembly 246 may be used to position the procedure rail 206 in a stowed position, in a procedure position, or any point therebetween. The stowed position of the robotic drive system 250 may be a position in which the rail adjustment assembly 246 is contracted and the procedure rail 206 is no longer above the patient support device 2 and/or the patient. The procedure position may include the procedure rail 206 being above the patient support device 2 and/or the patient, and with a device stack profile 24 (illustrated in FIGS. 8-10) aligned with, or approximately aligned with, the access point vasculature of the patient.

As illustrated in FIGS. 22-23, the procedure rail 206 is positioned for a pending procedure. The rail adjustment assembly 246 may be used to facilitate the positioning. The rail adjustment assembly 246 may include powered or manual mechanisms 212a, 212b, 212c, 212d, and/or 212e (illustrated in FIGS. 20 and 21). For example, the mechanisms may be powered and moved by control signals from a controller. The controller may actuate the powered mechanisms based on one or more signals received locally or remotely. Other embodiments may include unpowered or manual mechanisms 212a, 212b, 212c, 212d, and/or 212e (illustrated in FIGS. 20 and 21). Unpowered mechanisms may include friction mechanisms with sufficient friction as to hold the position of the respective mechanism (i.e., supporting the weight of the system) but not enough to overcome adjustment forces from a user. For example, a user may push, pull, or twist the procedure rail 206 to overcome the friction mechanisms, adjusting the position of the procedure rail 206. Alternatively, the mechanisms 212a, 212b, 212c, 212d, 212e (illustrated in FIGS. 20 and 21) may include braking controllable with local electrical or mechanical inputs. By selectively unlocking mechanisms and applying force to the procedure rail 206, a user may position the procedure rail 206 to a position appropriate for a pending procedure.

As described, the procedure rail 206, via the rail adjustment assembly 246, may be positioned next to, or above, the patient 1 and in a position appropriate for a pending procedure. With the described capabilities of the rail adjustment assembly 246, the procedure rail 206 may be positioned while avoiding anatomical features of the patient 1 (e.g., avoiding contact with the feet 264 illustrated in FIG. 23). As illustrated in FIG. 23, the procedure rail 206 may be translated along the rail longitudinal axis 220 toward the patient 1 or away from the patient 1 along the rail longitudinal axis 220.

The hubs 208a, 208b, 208c, 208d may be operatively coupled to the procedure rail 206 and may include drive mechanisms to translate the hubs 208a, 208b, 208c, 208d along the rail longitudinal axis 220. For example, in some embodiments, the hubs 208a-d may translate along a drive surface 211 of the procedure rail.

FIG. 23 depicts a cartesian coordinate frame. The cartesian coordinate frame includes an ordered triplet of lines X, Y, Z illustrating directional orientation of three-dimensional space. The directional orientation of the cartesian coordinate frame includes an X direction, a Y direction, and a Z direction corresponding to the ordered triplet of lines X, Y, Z respectively. The X direction, Y direction, and Z direction are mutually orthogonal. As shown in FIG. 23, with reference to the procedure rail 206, the X direction corresponds to a width in a horizontal orientation, the Y direction corresponds to a height in a vertical orientation, and the Z direction corresponds to a length in a horizontal orientation.

In some embodiments, the drive surface 211 may be a generally vertical drive surface. For example, the drive surface 211 may be a generally planar surface oriented in a vertical plane, for example, relative to a ground surface or a top surface of a patient support table which may be described as being oriented in a horizontal plane. The vertical plane may extend perpendicularly to the ground surface or top surface of the patient support table. The vertical plane may be a Y-Z plane with reference to the illustrated cartesian coordinate system. The patient support table 2 may be oriented in a horizontal plane (for example, an X-Z plane with reference to the illustrated cartesian coordinate system). In some embodiments, the drive surface 211 may be oriented along a generally vertical plane, which may be at an angle of less than about 5 degrees or less than about 10 degrees from a vertical plane (for example, about the Z-axis or about the axis 220). In some embodiments, the drive surface my be oriented at an angle of less than about 15 degrees, less than about 20 degrees, less than about 25 degrees, or less than about 30 degrees from a vertical plane (for example, about the Z-axis or about the axis 220). The foregoing angles between the plane of the drive surface 211 and the vertical plane may be dihedral angles about the Z-axis or about the longitudinal axis 220.

In some embodiments, during performance of a procedure, the procedure rail 206 may be oriented so that the longitudinal axis 220 extends partially vertically downward from a proximal end of the procedure rail 206 to distal end of the procedure rail 206 during a procedure. In other words, a proximal end of the procedure rail 206 can be positioned vertically (in the Y direction) above the distal end of the procedure rail 206 (for example, to provide a desired device stack profile).

Examples of drive mechanisms utilized by the hubs 208a, 208b, 208c, 208d may include a rack and pinion system, lead screw system, ball bearing based system, or any other suitable mechanism known in the art. The first hub 208a may be operatively coupled to a guidewire, a catheter, or another interventional device. The second hub 208b may be operatively coupled to a guidewire, catheter, or another interventional device. The third hub 208c may be operatively coupled to a guidewire, a catheter, or another interventional device. The fourth hub 208d may be operatively coupled to a guidewire, a catheter, or another interventional device. Some embodiments may include more than four hubs 208a, 208b, 208c, 208d, and the additional hubs may be operatively coupled to, respectively, a guidewire, a catheter, or another interventional device. The hubs 208a, 208b, 208c, 208d may include rotating hemostasis valves (referred to as RHV) 216a, 216b, 216c, 216d, respectively. RHV 216a, 216b, 216c, 216d may be used to operatively couple interventional devices (e.g., a catheter, or a guidewire) to respective hubs 208a, 208b, 208c, 208d. RHV 216a, 216b, 216c, 216d may include the capability to control of fluids or aspiration to each respective interventional device.

FIG. 23 may illustrate the embodiment of a robotic device drive system 250 in a procedure configuration. The first hub 208a may be operatively coupled to a first catheter (not shown) via the first RHV 216a, the second hub 208b may be operatively coupled to a second catheter (not shown) via the second RHV 216b, the third hub 208c may be operatively coupled to a third catheter (not shown) via the third RHV 216c, and the fourth hub 208d may be operatively coupled a guidewire (not shown) via the fourth RHV 216d (e.g., a guidewire hub). Each catheter and guidewire may be coupled to their respective hub at the proximal end of the catheter or guidewire forming a device stack profile 24 as illustrated in FIG. 8-10. Each RHV 216a, 216b, 216c, 216d may also contain mechanisms to rotate (as described for FIG. 11) or deflect the device as desired and may be connected to fluid delivery (e.g., contrast, saline, etc.) or suction lines (e.g., aspiration). The fluid delivery or suction lines 222a, 222b, 222c (which may be constructed with flexible material) are illustrated in FIG. 23. Line 222a connects the RHV 216a to junction point 219. Line 222b connects the RHV 216b to junction point 219. Line 222c connects the RHV 216c to junction point 219. Junction point 219 supports lines 222a, 222b, 222c and line lead 209. Line lead 209 (which may be constructed with flexible material) may include a plurality of fluid lines that separate at the junction point 219 into fluid lines 222a, 222b, 222c. As such, RHV 216a, 216b, 216c are independently connected to the fluidics system 200. In certain embodiments, an additional line may couple the RHV 216d to the junction point 219.

In certain embodiments, contrast fluid, saline, or and/or aspiration can be independently supplied to the RHV 216a, 216b, 216c of each respective hub, from the fluidics system 200, and as such, independently to the interventional devices coupled to RHV 216a, 216b, 216c. A guidewire coupled to the fourth hub 208d may be inserted within a catheter (which may be a third catheter in an interventional device assembly) coupled to the third hub 208c. The catheter coupled to the third hub 208c may be inserted into a catheter (which may be a second catheter in an interventional device assembly) coupled to the second hub 208b. The catheter coupled to the second hub 208b may be inserted into a catheter (which may be a first catheter in an interventional device assembly) coupled to the first hub 208a. As such, the outer diameter of the guidewire (not shown) would be less than inner diameter of the third catheter (not shown), the outer diameter of the third catheter (not shown) would be less than the inner diameter of the second catheter (not shown), and the outer diameter of the second catheter (not shown) would be less than the inner diameter of the first catheter (not shown). The described device stack profile (including the guidewire of the fourth hub 208d) can be manipulated by the robotic device drive system 250 for procedural requirements. For example, the distal end of the first catheter (not shown) may be axially adjusted, with respect to the device stack profile, by the first hub 208a advancing distally or retracting proximally along the procedure rail 206. The distal end of the second catheter (not shown) may be axially adjusted, with respect to the device stack profile, by the second hub 208d advancing distally or retracting proximally along the procedure rail 206. The distal end of the third catheter (not shown) may be axially adjusted, with respect to the device stack profile, by the third hub 208c advancing distally or retracting proximally along the procedure rail 206. The distal end of the guidewire (not shown) may be axially adjusted, with respect to the catheter stack, by the fourth hub 208d advancing distally or retracting proximally along the procedure rail 206. Contrast, saline, any other fluid, or aspiration can be supplied to the first, second and third catheter at any adjusted position.

Although FIGS. 19-24 show a standalone fluidics system in fluid communication with one or more hubs that may longitudinally translate along procedure rail 206, it can be appreciated that a backside 207 of the procedure rail 206 may include a fluidics system such that the fluidics system is integrated into the robotic drive system 250 and is movable with the rail assembly.

FIG. 25 illustrates an embodiment of a robotic device drive system 270. The robotic device drive system 270 can include a rail 4, one or more robotic arms 226a-e, and a fluidics system 200. For example, for certain procedures (e.g., neurovascular procedures), the robotic device drive system 270 may include a first robotic arm 226a, a second robotic arm 226b, a third robotic arm 226c, a fourth robotic arm 226d, and a fifth robotic arm 226e. Alternatively, a diagnostic system may include a first robotic arm. Alternatively, a supra-aortic access system may include a first robotic arm 226a, a second robotic arm 226b, a third robotic arm 226c, and optionally a fourth robotic arm 226d.

The first robotic arm 226a, the second robotic arm 226b, the third robotic arm 226c, the fourth robotic arm 226d, and the fifth robotic arm 226e may be operatively coupled to interventional devices (e.g., catheters, guidewires, etc.) as described herein. For example, the first robotic arm 226a, the second robotic arm 226b, the third robotic arm 226c, the fourth robotic arm 226d, and the fifth robotic arm 226e may include RHV 216a, 216b, 216c, 216d as described with respect to FIGS. 23 and 24. The first robotic arm 226a, the second robotic arm 226b, the third robotic arm 226c, the fourth robotic arm 226d, and the fifth robotic arm 226c may be functionally attached to a rail 4, which is attached to an elongate side 28 of the patient support device 2.

The robotic device drive system 270 may include at least some of the capabilities of the robotic device drive system 100 described for FIGS. 1, 2, and 5-10. For example, the first robotic arm 226a, which may be an introducer arm, may move into a position adjacent or proximate to access point vasculature (e.g., femoral artery, carotid artery, radial artery, etc.) of the patient 1. The first robotic arm 226a may translate along the rail 4 via a drive mechanism in communication with the rail 4. Examples of drive mechanisms that may move the first robotic arm 226a and/or any of the other robotic arms described herein include, but are not limited to: a rack and pinion system, a lead screw system, a ball bearing based system, a magnetic drive system, or any other suitable mechanism known in the art.

The first robotic arm 226a can translate or move along the rail 4 via a first robotic arm bottom portion 6a (illustrated in FIGS. 2, 6, and 7) and its associated drive mechanism. The bottom portion 6a (illustrated in FIGS. 2, 6, and 7) of the first robotic arm 226a can be operatively coupled to the first robotic arm intermediate portion 8a (illustrated in FIGS. 2, 6, and 7), which is attached to the first robotic arm head portion 10a (illustrated in FIGS. 2, 6, and 7). This configuration moves along the rail 4, ultimately along the elongate side 28 (illustrated in FIG. 2) of the patient support device 2 to a position predefined for the pending procedure.

The second robotic arm 226b can have a second robotic arm bottom portion 6b (illustrated in FIGS. 2, 6, and 7), second robotic arm intermediate portion 8b (illustrated in FIGS. 2, 6, and 7), and second robotic arm head portion 10b (illustrated in FIGS. 2, 6, and 7). The second robotic arm 226b can move along the rail 4, similar to the first robotic arm 226a as described above, to a position predefined for the pending procedure.

The third robotic arm 226c can have a third robotic arm bottom portion 6c (illustrated in FIGS. 2, 6, and 7), third robotic arm intermediate portion 8c (illustrated in FIGS. 2, 6, and 7), and third robotic arm head portion 10c (illustrated in FIGS. 2, 6, and 7). The third robotic arm 226c can move along the rail 4, similar to the first robotic arm 226a as described above, to a position predefined for the pending procedure.

The fourth robotic arm 226d can have a fourth robotic arm bottom portion 6d (illustrated in FIGS. 2, 6, and 7), fourth robotic arm intermediate portion 8d (illustrated in FIGS. 2, 6, and 7), and fourth robotic arm head portion 10d (illustrated in FIGS. 2, 6, and 7). The fourth robotic arm 226d may move along the rail 4, similar to the first robotic arm 226a as described above, to a position predefined for the pending procedure.

The fifth robotic arm 226e can have a fifth robotic arm bottom portion 6e (illustrated in FIGS. 2, 6, and 7), fifth robotic arm intermediate portion 8e (illustrated in FIGS. 2, 6, and 7), and fifth robotic arm head portion 10c (illustrated in FIGS. 2, 6, and 7). The fifth robotic arm 226c may move along the rail 4, similar to the first robotic arm 226a method described above, to a position predefined for the pending procedure. The robotic device drive system 270 may be fluidly connected to the fluidics system 200 as described herein. Fluidics lines 224a, 224b, 224c may connect to the hubs (not shown) of the second robotic arm 226b, the third robotic arm 226c, and the fourth robotic arm 226d, respectively. Fluidics lines 224a, 224b, 224c may be used to supply (i.e., administer) contrast, saline, any other fluid, or aspiration to the hub coupled to each arm 226b, 226c, 226d. The described robotic device drive system 270 can manipulate and position interventional devices (e.g., catheters, guidewires, etc.) as described for the robotic device drive system 100 in FIGS. 1, 2, and 5-11. Additionally, contrast, saline, any other fluid, or aspiration may be supplied to the interventional devices during procedures.

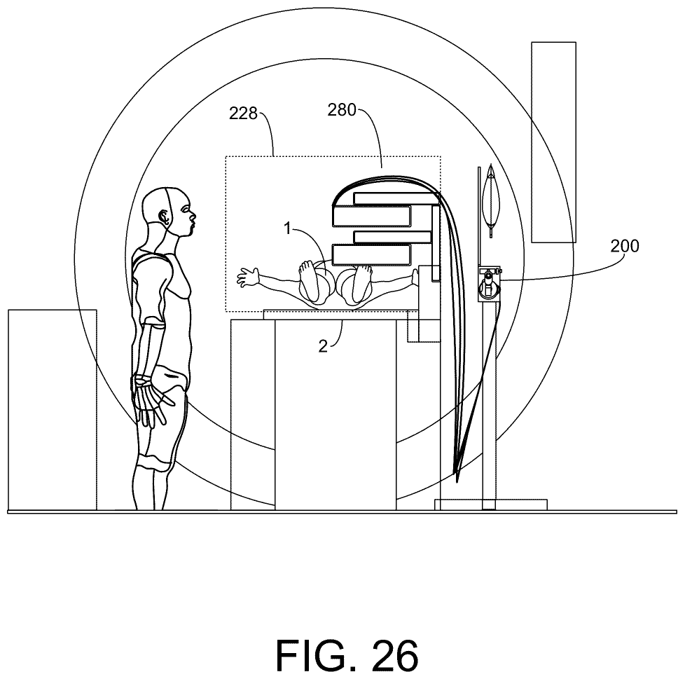

FIG. 26 illustrates a sterile zone 228 with respect to a robotic device drive system embodiment 280 and a standalone fluidics system 200. The robotic device drive system 280 may be representative of the robotic device drive system 250 described in FIGS. 20-24, or the robotic device drive system 270 described in FIG. 25. As illustrated, the robotic device drive system 280 used in a procedure with a standalone fluidics system 200, may be within the sterile zone 228 while the fluidics system 200 remains outside of the sterile zone 228. As such, the fluidics system 200 may not be subject to the draping requirements that may be required within the sterile zone 228. Eliminating draping requirements may increase efficiency of performing maintenance upon the fluidics system 200 (e.g., cleaning or replacing filters, etc.), replacing fluids or fluid reservoirs, or any other activity which require a user/technician to physically touch the fluidics system 200.

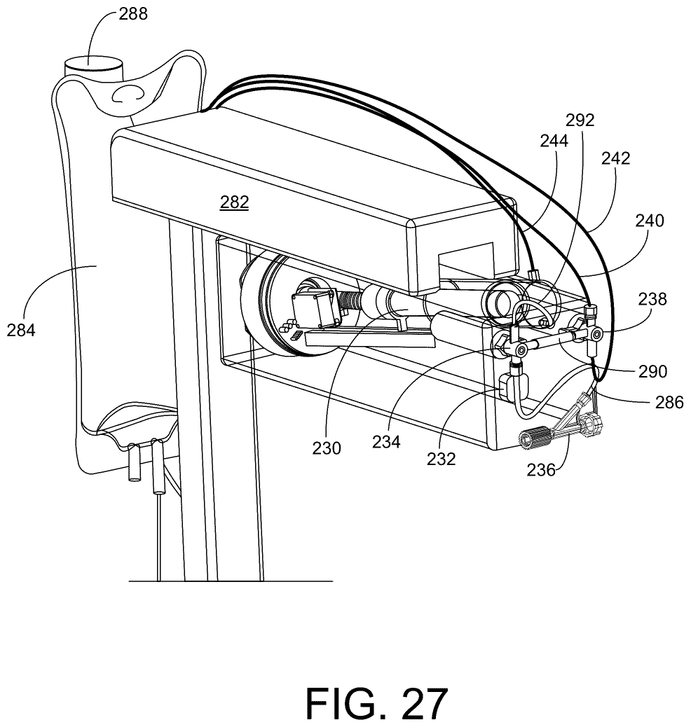

FIG. 27 illustrates an embodiment of a robotic arm 282 (shown at least partially transparent for describing internal components) defining one or more cavities for receiving fluidics components or systems therein. The robotic arm 282 includes a contrast injection pump 230, a first robotically actuated valve 234, a second robotically actuated valve 238, a fluid reservoir 284, a sensor 232, and a rotating hemostasis valve (referred to as RHV) 236. The reservoir 284 may be secured to a support 288. The RHV 236 may include a hemostasis valve. The RHV 236 may fluidly couple to an interventional device (e.g., a catheter) and may receive fluid (e.g., saline, contrast, or any other fluid) or aspiration from line 286. Line 286 may be connected to the output port of the first robotically actuated valve 234. The first robotically actuated valve 234 may include inputs connected to an output line 292 from the contrast injection pump 230, and an output line 290 from the second robotically actuated valve 238. The contrast injection pump 230 may receive contrast from an input line 244 coupled to a contrast source. Actuating the first robotically actuated valve 234 may put in fluid communication the input (e.g., contrast from the contrast injection pump 230) of line 292 to the output line 286, or the input of the line 290. Line 290 may be connected to the output of the second robotically actuated valve 238. Actuating the second robotically actuated valve 238 fluidly connects line 290 to either line 242, which may supply aspiration, or line 240, which may supply a pressurized fluid (e.g., saline from a peristaltic pump (not shown)). As such, control of the first robotically actuated valve 234 and the second robotically actuated valve 238 allows selection of line 292, line 240, or line 242 to be communicated to the line 286 connected to the RHV 236. The RHV 236 (including a hemostasis valve) allows communication of line 286 to the interventional device (e.g., a catheter) couple to the RHV 236. The described fluidics system of robotic arm 282 may be used with any robotic arm described herein (e.g., robotic arms 14, 16, 18, 20, 22 of FIGS. 1, 2, 6-10, and 12, robotic arms 50, 52, 54, 56 of FIG. 13, hubs 208a, 208b, 208c, 208d of FIGS. 22-24, and robotic arms 226a, 226b, 226c, 226d, 226c of FIG. 25). Using the described fluidics system with robotic arms or hubs of embodiments of FIGS. 19-26, in some instances, may replace the standalone fluidics system 200.

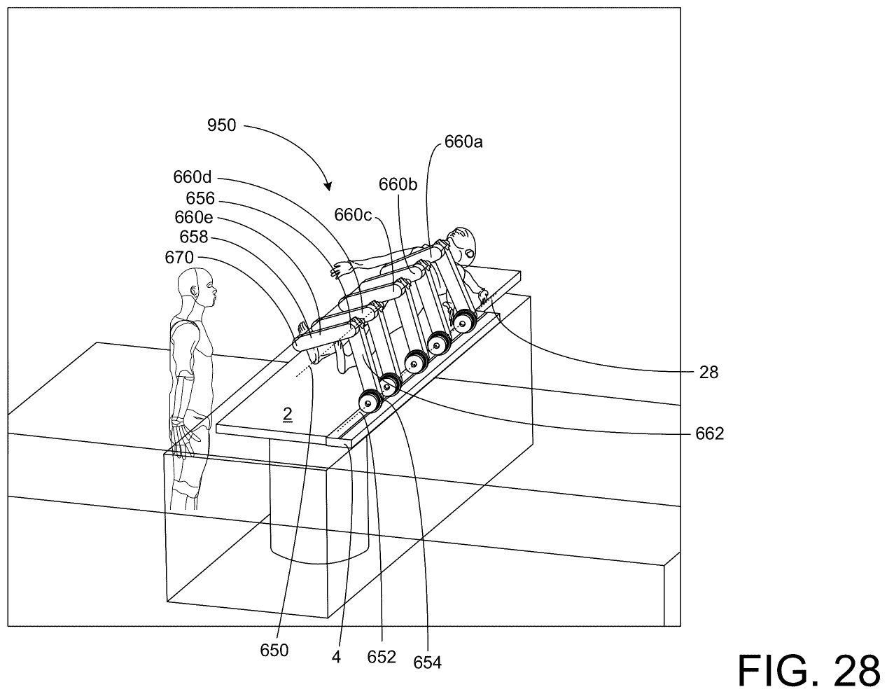

FIG. 28 illustrates a robotic device drive system 950 in an expanded configuration or procedure configuration. The robotic device drive system 950 may include a rail 4 and one or more robotic arms 660a-d. For example, for certain procedures (e.g., neurovascular procedures), the robotic device drive system 950 may include a first robotic arm 660a, a second robotic arm 660b, a third robotic arm 660c, a fourth robotic arm 660d, and a fifth robotic arm 660c. Alternatively, a diagnostic system may include a first robotic arm 660a. Alternatively, a supra-aortic access system may include a first robotic arm 660a, a second robotic arm 660b, a third robotic arm 660c, and optionally a fourth robotic arm 660d. Any number of robotic arms may be used for the intended purpose or procedure.

The first robotic arm 660a, the second robotic arm 660b, the third robotic arm 660c, the fourth robotic arm 660d, and the fifth robotic arm 660e may be operatively coupled to interventional devices (e.g., catheters, guidewires, etc.) as described herein. For example, one or more of the first robotic arm 660a, the second robotic arm 660b, the third robotic arm 660c, the fourth robotic arm 660d, and the fifth robotic arm 660e may include respective RHVs (such as RHV 216a, 216b, 216c, 216d as described for FIGS. 23 and 24). The first robotic arm 660a, the second robotic arm 660b, the third robotic arm 660c, the fourth robotic arm 660d, and the fifth robotic arm 660e may be operatively coupled to a rail 4, which is attached to an elongate side 28 of the patient support device 2.

Although the robotic device drive system 950 may include at least some of the capabilities of the robotic device drive system 100 described for FIGS. 1, 2, and 5-10, the robotic drive system 950 includes dissimilar robotic arm mechanisms. Instead of using robotic arm portions which translate with respect to one another, the robotic arm drive system 950 uses arm portions which can rotate to achieve a device stack profile 24 as shown in FIG. 8. For example, the fifth robotic arm 660e may translate along the rail 4 via a mechanism 654 in communication with the rail 4. Examples of drive mechanisms that may drive the mechanism 654 of the fifth robotic arm 660e include, but are not limited to: a rack and pinion system, a lead screw system, a ball bearing based system, a magnetic drive system, or any other suitable mechanism known in the art. Further, the mechanism 654 of the robotic arm 660e may include a rotational capability to rotate the base portion 662 of the arm 660e about the axis 652. Additionally, the robotic arm 660e may include a joint 656 between the base portion 662 and the upper portion 658. Joint 656 may allow the rotation of the upper portion 658 of the robotic arm 660e with respect to the base portion 662 about the axis 650. As such, the fifth robotic arm 660e can adjust the position of the end 670 of the upper portion 658, which may be connected (with devices described herein) to an interventional device, to achieve a desired position of an attached interventional device. One or more of the other robotic arms 660a, 660b, 660c, 660d may include an upper portion (as described for upper portion 658) and a base portion (as described for base portion 662) manipulatable by mechanisms similar to mechanisms 654, 656 of the fifth arm 660c.

The robotic device drive system 950 may include or may be fluidly connected to fluidics system as described herein. Although using dissimilar mechanisms (e.g., rotational instead of translational), the described robotic device drive system 950 can manipulate and position interventional devices (e.g., catheters, guidewires, etc.) as described for the robotic device drive system 100 in FIGS. 1, 2, and 5-11. Additionally, contrast, saline, any other fluid, or aspiration may be supplied to the interventional devices during procedures as described for other robotic drive systems herein.

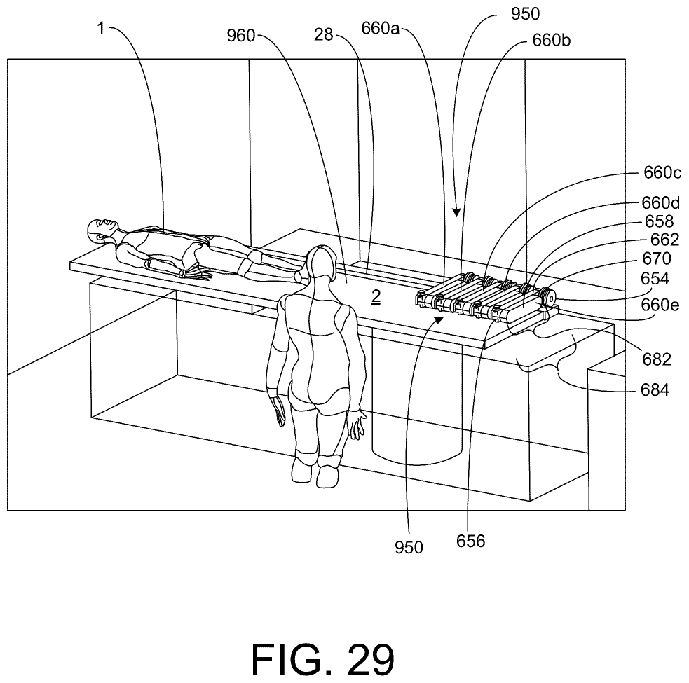

FIG. 29 illustrates the robotic drive system 950 in a contracted configuration with the robotic arms 660a-e segregated to an end of the patient support device 2, opposite of the patient 1. The contracted configuration of the fifth robotic arm 660e may include the length 682 of upper portion 658 and the length 684 of the base portion 662 parallel to one another. In other embodiments, the length 682 of the upper portion 658 and the length 684 of the base portion 662 may at least partially overlap in the contracted configuration. In some embodiments, the length 682 of upper portion 658 and the length 684 of the base portion 662 can be parallel to and above the lateral surface 960 of the patient support device 2 in the contracted configuration. Additionally, the end 670 of the upper portion 658 can be rotated to a point nearest the mechanism 654. Further, the contracted configuration of the fifth robotic arm 660e may include the length 682 of upper portion 658 and the length 684 of the base portion 662 substantially on the same side of mechanism 654. Robotic arms 660a, 660b, 660c, 660d may also be configured in this way when the robotic drive system 950 is in the contracted configuration.

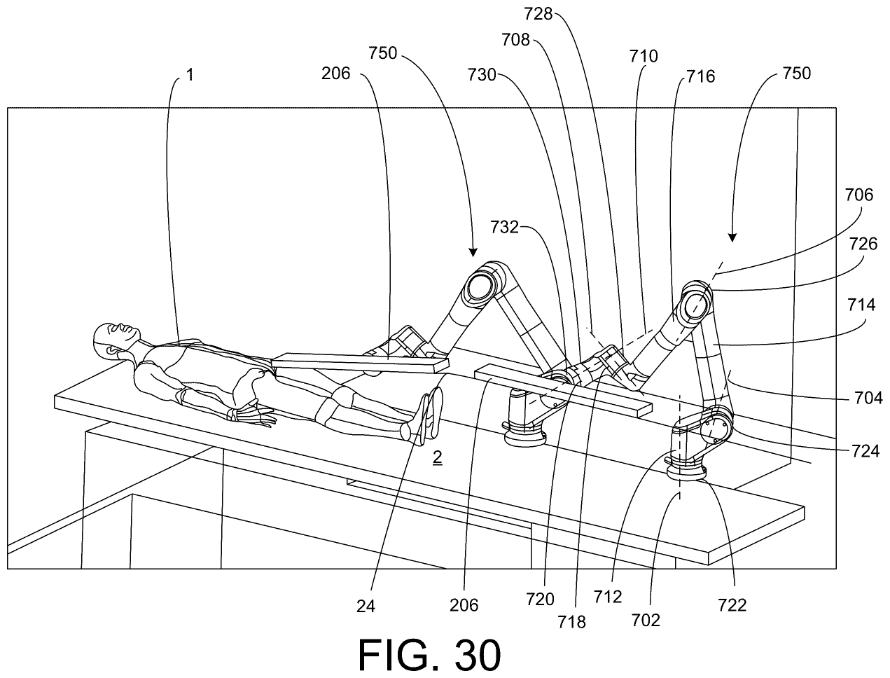

FIG. 30 illustrates another robotic drive system 750. The robotic drive system 750 can include a procedure rail 206 and one or more arm portions. For example, a robotic drive system 750 can include a first arm portion 712, a second arm portion 714, a third arm portion 716, a fourth arm portion 718, and a fifth arm portion 720. Although robotic drive system 750 is shown with five arm portions, one of skill in the art will appreciate that an arm portion, one or more arm portions, a plurality of arm portions, etc. may be used to achieve the desired movement, range of motion of the system 750, flexibility, etc. One or more arm portions may be of rigid construction and/or one or more arm portions may be flexible.

The procedure rail 206 may include one or more hubs, such as hubs 208a-d and mechanisms as described for FIGS. 22-24. The first arm portion 712 may be operatively coupled to the patient support device 2 by a first joint 722, which can allow rotation of the first arm portion 712 about the pivotal axis 702 of the first joint 722 (i.e., may allow yaw adjustments of the first arm portion 712). Further, the first arm portion 712 may be operatively coupled to the second arm portion 714 by a second joint 724, which allows rotation of the second arm portion 714 about the pivotal axis 704 of the second joint 724 (i.e., may allow pitch adjustments of the second arm portion 714). The second arm portion 714 may be operatively coupled to the third arm portion 716 by a third joint 726, which allows rotation of the third arm portion 716 about the pivotal axis 706 of the third joint 726 (i.e., allowing pitch adjustments of the third arm portion 716). The third arm portion 716 may be operatively coupled to the fourth arm portion 718 by a fourth joint 728, which allows rotation of the fourth arm portion 718 about the pivotal axis 708 of the fourth joint 728 (i.e., may allow roll adjustments of the fourth arm portion 718). The fourth arm portion 718 may be operatively coupled to the fifth arm portion 720 by a fourth joint 730, which allows rotation of the fifth arm portion 720 about the pivotal axis 710 (i.e., may allow roll adjustments of the fifth arm portion 720). The procedure rail 206 is coupled to the fifth arm portion 720 at interface 732 in such a way that rotation of the fifth arm portion 720 can rotate the procedure rail 206 about the axis 710. Interface 732 may also allow the procedure rail 206 to translate along the rail longitudinal axis 220 (for example, as illustrated in FIGS. 23 and 24) with respect to the interface 732. The robotic drive system 750, as described, may be capable of at least positioning the procedure rail 206 as described for the robotic drive system 250 for FIGS. 20-24.

It has been contemplated herein that two or more robotic drive systems (i.e., a dual assembly) may work in unison, in tandem, synchronously, asynchronously, or otherwise to perform certain procedures. As shown in FIG. 30, two robotic drive systems 750 may position two procedure rails 206 for a pending procedure. Although the hubs associated with one or more interventional devices may be operatively coupled to separate procedure rails, the interventional devices of each procedure rail may form a collective or combined device stack within a collective or combine device stack profile 24. Positioning two separate procedure rails 206 may be advantageous due to the fact that the configuration includes the capability of non-linear device stack profiles 24. Robotic drive systems (e.g., robotic drive system 250 from FIGS. 20-24) with a single procedure rail 206 may only be capable of linear device stack profiles.

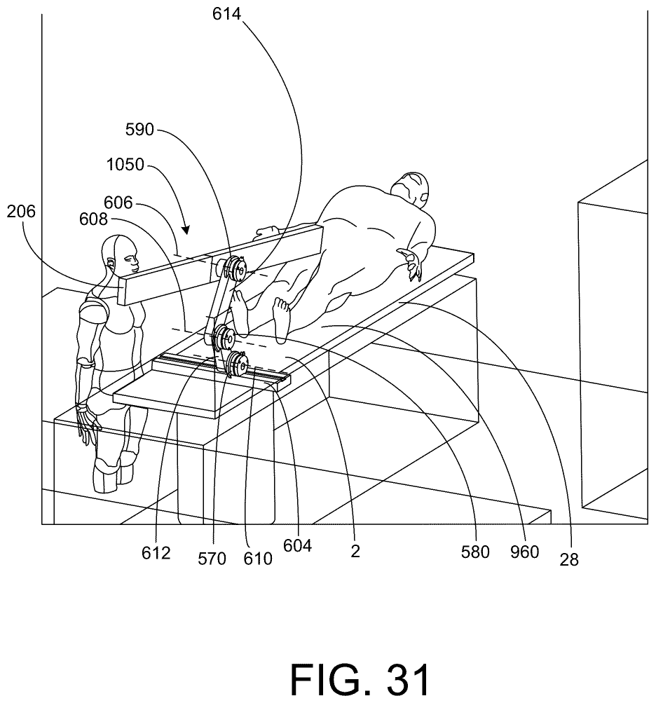

Several embodiments described herein utilize a rail 4 which is parallel to the elongate side 28 of the patient support device 2 (e.g., shown in FIGS. 12, and 28), but it has been contemplated herein that robotic drive systems may utilize a rail transverse to or perpendicular to the elongate side 28 of the patient support device 2. As shown in FIG. 31, a robotic drive system 1050 may include a rail 604 coupled to the patient support device 2 perpendicular to the elongate side 28, for example, on the lateral face 960 of the patient support device 2. Further, the robotic drive system 1050 may include a base portion 612, an upper portion 614, and a procedure rail 206. The procedure rail 206 may include one or more hubs 208a-d and mechanisms described for FIGS. 22-24. The base portion 612 may be operatively coupled to the rail 604 by a mechanism 570. The mechanism 570 may translate the base portion 612, and therefore the entire robotic drive system, along the rail 604 and along the axis 610. Additionally, the mechanism 570 may include the added capability of allowing the base portion 612 to rotate about the pivotal axis 610 of the mechanism 570 (i.e., may allow pitch adjustments of the base portion 612). The base portion 612 may be operatively coupled to the upper portion 614 by joint 580. Joint 580 may allow the upper portion 614 to rotate about the pivotal axis 608 of the joint 580 (i.e., may allow pitch adjustments of the upper portion). The upper portion 614 may be operatively coupled to the procedure rail 206 by interface 590. Interface 590 may allow the procedure rail 206 to rotate about the pivotal axis 606 of the interface 590 (i.e., may allow pitch adjustments of the procedure rail 206). Further, interface 590 may also allow the procedure rail 206 to translate along the rail longitudinal axis 220 (illustrated in FIGS. 23 and 24) with respect to the interface 590. The robotic drive system 1050, as described, may position the procedure rail 206 in a contracted (i.e., storage) configuration, an expanded configuration, or any point therebetween. The joints, mechanisms, and interfaces described with respect to FIGS. 28-31 may include rotary mechanisms, hinges, revolute joints, elbow joints, rotational joints, or any other appropriate joint or mechanism known in the art.

Several embodiments described herein utilize a rail 4 which is parallel to the elongate side 28 of the patient support device 2 (e.g., embodiments described with respect to FIGS. 1-10, 13, 25, 28 and 29) for translational movements, parallel to the elongate side 28 of patient support device 2, of the robotic arms and, thus, their respective interventional devices. It is contemplated that some embodiments may instead utilize robotic arms coupled to the elongate side 28 of the patient support device 2 at pre-determined positions or removably coupled such that the robotic arms may be movable to different positions along the elongate side of the patient support device 2. These described robotic arms may include a joint (e.g., a swing joint similar to the first joint 722 described in FIG. 30). As described for the embodiment of FIG. 30, the first joint 722 can allow rotation of the first arm portion 712 about the pivotal axis 702 of the first joint 722 (i.e., may allow yaw adjustments of the robotic arm). The robotic arms of the embodiments of FIGS. 1-10, 13, 25, 28 and 29 may achieve the same translational movements described for the translation of robotic arms along the rail 4 (rail 58 for FIG. 13) by coordinated movements of the robotic arms about the described joint that couples the robotic arms to the patient support device and the other joints and/or mechanism described for the robotic arms. The described robotic arm embodiments are coupled to respective interventional devices. The robotic arms of the embodiments described for FIGS. 1-10, 13, 25, 28 and 29 remain in a fixed yaw orientation with respect to the device stack profile (e.g., robotic arm lengths being perpendicular to device stack profile), but the modified robotic arm embodiments utilizing the described swing joint can interface with the device stack profile at various angles during the coordinated movements of the robotic arms about the swing joint and the other joints and/or mechanism described for the robotic arms. As such, it may be advantageous for the described robotic arms to include a mechanism (e.g., a hinge, a ball joint, a U-joint, etc.) within the interface with their respective interventional device and/or associated hardware (e.g., a hub) to allow the respective interventional device to remain aligned with the device stack during the described translational movements.

FIG. 32 illustrates a schematic of an embodiment of a standalone fluidics system 300 (e.g., fluidics system 200 illustrated in FIGS. 19-26). In general, the fluidics system 300 includes a cassette that can couple a plurality of fluid sources and/or sinks to a plurality of interventional devices. A plurality of fluid lines may extend between a source or sink and the cassette for coupling to different interventional devices. For other sources and sinks, a single fluid line may extend between the source or sink and the cassette and may split within the cassette to connect to different interventional devices. In certain embodiments, the cassette can include connection arrays formed of connections from a plurality of fluid sources and/or sinks for coupling to a single interventional device (for example, in a row or column). Each connection array can couple to a tubing set having a tube corresponding to each connection in the connection array.

The cassette 341 may be a self-contained unit comprising a housing having a plurality of valves, tubing and connectors as described below. A first connector array comprises a plurality of releasable connectors such as luer connectors, for placing the cassette in fluid communication with complementary connectors in fluid communication with sources of aspiration and at least one or two or more fluids. A second connector array is configured for releasable connection to a tubing set configured to extend between the cassette and at least one or two or three interventional devices.

The cassette 341 thus forms a bridge module that, when assembled, resides between the various fluid and vacuum sources, and the corresponding interventional devices. The cassette 341 may be configured for a single use, or may be resterilizable and reusable.

As shown in FIG. 32, the system 300 may include a first fluid source 310a and a second fluid source 310b. Fluid flow from the first source 310a is directed through a one way check valve 314 and on to a high pressure pump 352, which may be a syringe pump, high pressure positive displacement pump, contrast injection pump, etc. The fluid from the first fluid source 310a may be a contrast solution which is preferably injected under high pressure.

Fluid flow from the syringe pump is directed into a cassette 341, which may include a plurality of valves, manifolds, and/or connectors. Within the cassette 341, the fluid flow may split along a plurality of branches 318b to a plurality of connectors 317b (for example, four connectors 317b as shown in FIG. 4.) for coupling with different interventional devices. The cassette may include a valve 316b (e.g., a ball valve) with each branch 318b upstream of the connector 317b. In certain embodiments, any of the valves 316a or 316b can be a ball valve, a stopcock valve, a rotary valve, a solenoid valve, or any other suitable valve.

Fluid flow from the second fluid source 310b may be directed into a plurality of branches 318c to a plurality of pumps 334 (for example, four pumps 334 as shown in FIG. 32), such as peristaltic pumps or rotary piston pumps. Each pump 334 can drive the fluid (for example, saline) under pressure from the second fluid source 310b to a unique connector 317c for each interventional device within the cassette 341.

The system further includes an aspiration canister 340 in communication with an upstream side of a filter 344. A downstream side of the filter 344 is in communication with a vacuum pump 342. The aspiration canister receives fluid from the cassette 341 which includes a plurality of connectors 317a each being configured to couple to a unique interventional device. A unique valve 316a (at least two, and four in the illustrated example) may be positioned upstream of each connector 317a. Each unique valve 316a may be positioned along a branch 318a.

In certain embodiments, one or more connector arrays 346 may be arranged, each connector array 346 configured to couple an interventional device. For example, a connector array 346 is indicated by dashed lines in FIG. 4. As shown in FIG. 32, the connector array 346 can include a connector 317a, a connector 317b, and a connector 317c. As shown in FIG. 32, the array 346 may be organized with all connectors facing in the same direction on a common plane, such as a linear row.

The connector array 346 can releasably couple to a tubing set 343 including an aspiration tube 354, a first fluid tube 355, and a second fluid tube 356. In some embodiments, the connectors 317a, 317b, and 317b can be Luer lock connectors. The aspiration tube 354 can couple to the connector 317a of the array 346 by way of a complementary connector 317d for aspiration from the interventional device to the aspiration container. The first fluid tube 355 can couple to the connector 317b of the array 346 by way of a complementary connector 317e to provide fluid flow from the first fluid source 310a to the interventional device. The second fluid tube 356 can couple to the connector 317c of the array 346 by way of a complementary connector 317f to provide fluid flow from the second fluid source 310b to the interventional device. The tubes 354, 355, and 356 may be joined together over a majority of their lengths. The tubes 354, 355, and 356 can each have a length of at least about three or four feet, and in certain embodiments between about 6 feet and about 8 feet.

As shown in FIG. 32, the tubing set 343 includes a line branch point 308 (e.g., a two to one or a three to one wye) that can provide fluid communication between the interventional device and the tube 354, tube 355, and tube 356. The line branch point 308 may include Luer lock connectors or wye connectors that interface with complementary connectors on the tubing set. In certain embodiments, a one-way valve 345 may be positioned upstream of the branch point 308 and downstream of the cassette 341 along the flow path of the second fluid.

In certain embodiments, the system 300 (or other systems described herein) can direct the flow of the second fluid (for example, saline) using two different flow modes. In a low flow drip mode, a flow rate of about 1-2 drips per second or 3-6 mL/min may be provided, for example, by the pumps 334. In some embodiments, a low flow mode rate of 1-8 mL/min may be provided. Each catheter coupled to the system may experience a different fluid resistance as described herein.

The pumps, for example pumps 334, can be operated to provide the same flow rate in each catheter. In certain embodiments the fluid pressure within the catheter can be at least about 330 mmHg or 6.5 psi. This pressure may be enough to overcome arterial pressure while delivering the desired drip rate. In certain embodiments, the pressure within the catheter can be greater than 330 mmHg. In certain embodiments, the delivered fluid volume can be at least about 1 liter over the length of a procedure. In some embodiments, the fluid volume can be up to 2 liters.

In a high flow flush mode, all of the fluid lines may be flushed to remove air. The flow rate can be between 100-1000 ml/min. The fluid pressure may be between 5-10 psi. The volume delivered can be between 0.5-1 liters per procedure. Volume may depend on tubing length and diameter. In some embodiments, the high flush flow rate is at least about 20 times and in some cases between 30 to 150 times the low flow drip mode flow rate.

In certain embodiments, the first fluid (for example, contrast solution) can be provided at a flow rate of between 3-8 L/s (for example, about 4 mL/s), for example, by the pump 352. In certain embodiments, the flow rate can be up to about 8 mL/s. In other embodiments, the flow rate can be up to about 20 mL/s. In certain embodiments, the first fluid can be provided with a pressure of about 400 psi for a flow rate of about 4 mL/s. The amount of pressure needed may depend on flow rate and flow restriction of the fluid path. The pressure may increase proportionally with the flow rate for higher flow rates. In certain embodiments, the pressure may be up to 1200 psi.

In certain embodiments, the high pressure pump, such as pump 352, can provide a delivered volume of between 5-15 mL per high pressure injection. In certain embodiments, the pump can provide the 5-15 mL per high pressure injection in increments of about 1 mL per puff. In certain embodiments, the second fluid source can provide a total volume of about 200 mL per procedure. In certain embodiments, the syringe pump is sized to hold at least about 150 mL or 200 mL so as to provide uninterrupted flow throughout the procedure without the need to add additional contrast solution. In other embodiments, the second fluid source can provide a total volume of between 150-250 mL per procedure.

In certain embodiments, the flow rate may vary depending upon the anatomical location at the distal end of the catheter. For example, within the aortic arch, the flow rate may be about 20 mL/s. A total delivered volume of about 25 mL may be infused in the aortic arch. Within the common carotid artery, the flow rate may be about 20 mL/s. A total delivered volume of 12 mL may be infused in the common carotid artery. Within the subclavian artery, the flow rate may be about 6 mL/s. A total delivered volume of about 15 mL may be infused in the subclavian artery. Within the internal carotid artery, the flow rate may be about 6 mL/s. A total delivered volume of about 8 mL may be infused in the internal carotid artery. Within the external carotid artery, the flow rate may be about 3 mL/s. A total delivered volume of about 6 mL may be infused in the external carotid artery. Within the vertebral artery, the flow rate may be about 6 mL/s. A total delivered volume of 8 mL may be infused in the vertebral artery.

In certain embodiments, a motor may be provided to drive the high pressure pump, such as pump 352, which can be controlled with a position and velocity control loop using a potentiometer as a measurement to close the loop. In certain embodiments, current control may be applied to provide approximate pressure limiting. In certain embodiments, the second fluid can be a contrast solution such as Omnipaque 300, Omnipaque 350, or Visipaque 320.

In certain embodiments, a vacuum pump, such as pump 342, can provide a pressure of about −29.5 inHg or up to −29.5 inHg (−999 mbar). In certain embodiments, tubing used for aspiration can have an inner diameter of 0.11 inches (about 2.8 mm). In certain embodiments, the volume of the aspiration container, such as canister 340, can be at least about 0.5 L. In certain embodiments, the volume of the aspiration container can include about 0.5 L for blood and additional volume for a saline flush. In certain embodiments, the aspiration container can have a volume between 0.25-0.75 L. In certain embodiments, the vacuum pump can be configured to operate to additionally provide a low pressure/flow setting to assist a flushing process as it may be desirable that an aspiration line is full of saline at all times (except when aspirating a clot). In certain embodiments, a separate pump may be provided for the low pressure/flow setting.

In certain embodiments, one or more of the hubs (e.g., hubs 208a-d) and/or interventional devices described herein may be magnetically driven. For example, in certain embodiments, one or more hubs may be magnetically driven along a drive table or procedure rail (e.g., procedure rail 206).

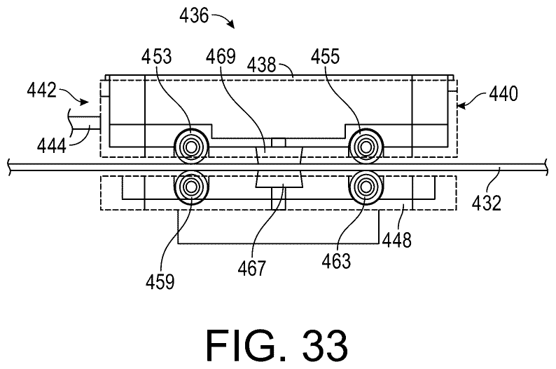

Referring to FIG. 33, hub 436 may represent any of the hubs previously described. Hub 436 includes a housing 438 which extends between a proximal end 440 and a distal end 442. An interventional device 444, which could be any of the interventional devices disclosed herein, extends distally from the hub 436 and into the patient (not illustrated). A hub adapter 448 or carriage acts as a shuttle by advancing proximally or distally along a track in response to operator instructions or controller manipulations. The hub adapter 448 includes at least one drive magnet 467 configured to couple with a driven magnet 469 carried by the hub 436. This provides a magnetic coupling between the drive magnet 467 and driven magnet 469 through the sterile barrier such that the hub 436 is moved across the top of the sterile barrier 432 in response to movement of the hub adapter 448 outside of the sterile field. Movement of the hub adapter is driven by a drive system carried, for example, by a drive table or procedure rail. The hub adapter may act as a robotic drive for an interventional device coupled thereto.

To reduce friction in the system, the hub 436 may be provided with at least a first roller 453 and a second roller 455 which may be in the form of wheels or rotatable balls or drums. The rollers space the sterile barrier apart from the surface of the driven magnet 469 by at least about 0.02 centimeters (about 0.008 inches) and generally no more than about 0.08 centimeters (about 0.03 inches). In some implementations, the space is within the range of from about 0.03 centimeters (about 0.010 inches) and about 0.041 centimeters (about 0.016 inches). The space between the drive magnet 467 and driven magnet 469 is generally no more than about 0.38 centimeters (about 0.15 inches) and in some implementations is no more than about 0.254 centimeters (about 0.10 inches) such as within the range of from about 0.216 centimeters (about 0.085 inches) to about 0.229 centimeters (about 0.090 inches). The hub adapter 448 may similarly be provided with at least a first hub adapter roller 459 and the second hub adapter roller 463, which may be positioned opposite the respective first roller 453 and second roller 455 as illustrated in FIG. 33.

### EXAMPLES

Example 1. A robotic device drive system comprising: a plurality of robotic arms arranged on a rail and configured to axially translate along the rail from a contracted configuration to an expanded configuration, wherein at least one of the plurality of robotic arms comprises: a fluidics system configured to be fluidly coupled to a hemostasis valve; and a catheter hub coupled to a catheter, the catheter hub comprising the hemostasis valve, wherein axial translation of the at least one of the plurality of robotic arms causes axial translation of the catheter.

Example 2. The robotic device drive system of any one of the preceding examples, but particularly Example 1, wherein the fluidics system further comprises: tubing that fluidly connects the hemostasis valve to a sink and a source, the source being stored within the at least one of the plurality of robotic arms; and a bubble removal system configured to be activated upon detection of bubbles in a portion of the tubing.

Example 3. The robotic device drive system of any one of the preceding examples, but particularly Example 2, wherein the fluidics system further comprises: a first valve in communication with a first port; a second valve in communication with a second port; and a third valve in communication with a third port, wherein the fluidics system is configured to selectively place the third port in communication with a portion of the catheter while simultaneously blocking the first port and the second port from communication with the portion of the catheter.

Example 4. The robotic device drive system of any one of the preceding examples, but particularly Example 3, wherein the fluidics system further comprises a source of vacuum, and the first port is configured to connect to the source of vacuum, the second port is configured to connect to a source of saline, and the third port is configured to connect to a source of contrast media.

Example 5. The robotic device drive system of any one of the preceding examples, but particularly Example 1, wherein the rail is coupled to an elongate side of a patient support device and the at least one of the plurality of robotic arms respectively comprises: a bottom arm portion configured to axially move along the rail; an intermediate arm adjustable from a first height relative to a lateral surface of the patient support device to a second height relative to the lateral surface of the patient support device; and a head portion configured to extend away from the intermediate arm portion and suspend above at least a portion of the patient support device.

Example 6. The robotic device drive system of any one of the preceding examples, but particularly Example 5, wherein the first height is a storage height and the second height is a procedure height.

Example 7. The robotic device drive system of any one of the preceding examples, but particularly Example 5, wherein the intermediate arm portion is height configurable between the first height and the second height to avoid contact of the at least of the plurality of robotic arms with one or more first patient body portions while another of the plurality of the plurality of robotic arms is attached to a first end of a patient introducer, the patient introducer being coupled to a second patient body portion at the second end.

Example 8. The robotic device drive system of any one of the preceding examples, but particularly Example 1, wherein the robotic device drive system is installed on an elongate side of a patient support device and the plurality of robotic arms comprise: a patient introducer arm; at least one guidewire arm; at least one guide catheter arm; at least one access catheter arm; and at least one procedure catheter arm.

Example 9. The robotic device drive system of any one of the preceding examples, but particularly Example 1, wherein: at least some of the plurality of robotic arms are independently adjustable in a vertical direction perpendicular to the rail; and at least some of the plurality of robotic arms are configured to move in tandem when axially translated along the rail from the expanded configuration to the contracted configuration.

Example 10. A robotic device drive control system comprising: a robotic arm configured to move axially along a rail, the robotic arm comprising: a catheter hub coupled to a catheter and configured to roll the catheter; and a valve system in communication with the catheter hub and configured to administer fluids to a hemostasis valve coupled to a portion of the catheter

Example 11. The robotic device drive control system of any one of the preceding examples, but particularly Example 10, further comprising an anti-buckling control configured to perform anti-buckling operations for the catheter.

Example 12. The robotic device drive control system of any one of the preceding examples, but particularly Example 10, wherein the robotic arm comprises a bottom arm portion, an intermediate arm portion and a head arm portion, the intermediate arm portion having a proximal end coupled to the bottom arm portion and a distal end coupled to the head arm portion, the head arm portion having a proximal end coupled to at least one portion of the intermediate arm portion and a distal end coupled to an interface to the catheter.

Example 13. The robotic device drive control system of any one of the preceding examples, but particularly Example 10, wherein the robotic arm is configured to move along the rail in a first horizontal direction that is substantially parallel to the rail and in a second vertical direction that is substantially perpendicular to the rail.

Example 14. A robotic device drive control system comprising: at least one processor; and a robotic arm comprising a catheter hub and a valve system in fluid communication with the catheter hub, the catheter hub comprising a hemostasis valve and the at least one processor configured to execute instructions to: move the robotic arm axially along a rail of a patient support device, roll a catheter coupled to the hemostasis valve of the catheter hub, and administer fluids through the valve system in fluid communication with the hemostasis valve coupled to a portion of the catheter.

Example 15. A robotic device drive control system comprising: at least one robotic arm operatively coupled to a rail, the at least one robotic arm comprising a fluidics system and a catheter hub; a controller configured to be in electrical communication with the fluidics system and the catheter hub; an adapter configured to couple both the fluidics system and the catheter hub to at least one catheter, wherein the adapter enables manipulation of the at least one catheter based on signals received from the controller.

Example 16. The robotic device drive control system of any one of the preceding examples, but particularly Example 15, wherein a first side of the adapter is in a sterile field and a second side of the adapter is in a nonsterile field.

Example 17. The robotic device drive control system of any one of the preceding examples, but particularly Example 15, further comprising: a camera coupled to the at least one robotic arm; a plurality of motors coupled to portions of the fluidics system and configured to control a plurality of ports and valves of the fluidics system; a hemostasis valve coupled to the catheter hub and the at least one catheter, wherein the controller is installed in the catheter hub and is configured to control the camera, the plurality of motors, the hemostasis valve, and the at least one catheter.

Example 18. The robotic device drive control system of any one of the preceding examples, but particularly Example 15, wherein the fluidics system further comprises: tubing that fluidly connects a hemostasis valve, in communication with the at least one catheter, to a sink and a source, the source being stored within the at least one robotic arm; and a bubble removal system configured to be activated upon detection of bubbles in a portion of the tubing.

Example 19. The robotic device drive control system of any one of the preceding examples, but particularly Example 18, wherein the fluidics system further comprises: a first valve in communication with a first port; a second valve in communication with a second port; and a third valve in communication with a third port, wherein the fluidics system is configured to selectively place the third port in communication with a portion of the at least one catheter while simultaneously blocking the first port and the second port from communication with the portion of the at least one catheter.

Example 20. The robotic device drive control system of any one of the preceding examples, but particularly Example 19, wherein the fluidics system further comprises a source of vacuum, and the first port is configured to connect to the source of vacuum, the second port is configured to connect to a source of saline, and the third port is configured to connect to a source of contrast media.

Example 21. A robotic device drive control system comprising: a first robotic arm comprising a first catheter hub and a first valve system in communication with the first catheter hub; a second robotic arm comprising a second catheter hub and a second valve system in communication with the second catheter hub; and a rail, wherein the first robotic arm and second robotic arm are operatively coupled to the rail, wherein the first catheter hub is configured to be in communication with a first hemostasis valve through a first interface, the first interface being configured to: receive movement control signals to move a first catheter associated with the first hemostasis valve, and receive fluid control signals for administering fluid from the first valve system to the first catheter.

Example 22. The robotic device drive control system of any one of the preceding examples, but particularly Example 21, wherein the second catheter hub is configured to be in communication with a second hemostasis valve through a second interface, the second interface being configured to: receive movement control signals to move a second catheter associated with the second hemostasis valve, and receive fluid control signals for administering fluid from the second valve system to the second catheter.

Example 23. The robotic device drive control system of any one of the preceding examples, but particularly Example 21, wherein the first robotic arm and the second robotic arm are configured to axially translate along the rail.

Example 24. The robotic device drive control system of any one of the preceding examples, but particularly Example 21, further comprising a rail adjustment assembly for positional adjustments of the rail.

Example 25. The robotic device drive control system of any one of the preceding examples, but particularly Example 21, wherein the first robotic arm and the second robotic arm are configured to axially translate along the rail from a contracted configuration to an expanded configuration or from an expanded configuration to a contracted configuration.

Example 26. The robotic device drive control system of any one of the preceding examples, but particularly Example 21, wherein the robotic device drive control system is configured to gain supra-aortic vessel access, the robotic device drive control system further comprising: a guidewire arm comprising a guidewire hub being configured to adjust an axial position of a guidewire and a rotational position of the guidewire, wherein the guidewire arm is operatively coupled to the rail, and wherein: the first catheter hub is configured to adjust an axial position of the first catheter; and the second catheter hub is configured to adjust an axial position of the second catheter and a rotational position of the second catheter, the second catheter hub being further configured to laterally deflect a distal deflection zone of the second catheter.

Example 27. The robotic device drive control system of any one of the preceding examples, but particularly Example 21, wherein: the first catheter hub is a guide catheter hub; and the second catheter hub is a procedure catheter hub.

Example 28. A robotic device drive control system comprising: a rail; a rail adjustment system configured to manipulate a position of the rail between a stored configuration to a procedure configuration; a fluidics system; and a plurality of catheter hubs operatively coupled to the rail and configured to be axially translated along a longitudinal axis of the rail, wherein at least one of the plurality of catheter hubs is in fluid communication with the fluidics system, the at least one of the plurality of catheter hubs comprising a hemostasis valve and a catheter in communication with the hemostasis valve.

Example 29. The robotic device drive control system of any one of the preceding example, but particularly Example 28, wherein the fluidics system is configured to selectively supply fluid or vacuum to the hemostasis valve.

Example 30. The robotic device drive control system of any one of the preceding example, but particularly Example 29, wherein the fluidics system is configured to selectively supply fluid from a saline source and a contrast source.

Example 31. The robotic device drive system of any one of the preceding examples, but particularly Example 28, wherein the plurality of catheter hubs comprise: at least one guidewire hub; at least one guide catheter hub; at least one access catheter hub; and at least one procedure catheter hub.

Example 32. A method of performing a thrombectomy with a robotic device drive system, the method comprising: translating a first robotic arm operatively coupled to a first catheter axially along a rail and toward an access point vasculature of a patient to introduce the first catheter into the access point vasculature of the patient; translating a second robotic arm operatively coupled to a second catheter positioned concentrically within a lumen of the first catheter axially along the rail; and selectively supplying one or more fluids to or aspirating fluids from to one or both of the first catheter and the second catheter by a fluidics system.

Example 33. The method of any one of the preceding examples, but particularly Example 32, further comprising translating axially along the rail a third robotic arm operatively coupled to a third catheter positioned concentrically within a lumen of the second catheter; and selectively supplying one or more fluids to or aspirating fluids from one or more of the first catheter, the second catheter, and the third catheter by the fluidics system.

Example 34. The method of any one of the preceding examples, but particularly Example 33, further comprising translating axially along the rail a fourth robotic arm operatively coupled to a guidewire positioned within a third lumen of the third catheter.

Example 35. The method of any one of the preceding examples, but particularly Example 32, further comprising translating axially along the rail a third robotic arm operatively coupled to a guidewire positioned within a second lumen of the second catheter.

Example 36. The method of any one of the preceding examples, but particularly Example 32, further comprising translating axially along the rail an introducer arm operatively coupled to a guide tube to position the guide tube near the access point vasculature, wherein the first catheter is positioned within a lumen of the guide tube.

Example 37. A method of performing a thrombectomy procedure with a robotic device system, the method comprising: positioning a rail to approximately align a device stack profile to an access point vasculature of a patient; translating a first hub operatively coupled to a first catheter axially along the rail and toward the access point vasculature to introduce the first catheter into the access point vasculature; translating axially along the rail a second hub operatively coupled to a second catheter positioned within a first lumen of the first catheter; and selectively supplying one or more fluids to or aspirating fluids from one or both of the first catheter and the second catheter by a fluidics system.

Example 38. The method of any one of the preceding examples, but particularly Example 37, wherein the rail is positioned by a rail adjustment assembly.

Example 39. The method of any one of the preceding examples, but particularly Example 37, further comprising translating axially along the rail a third hub operatively coupled to a third catheter positioned within a second lumen of the second catheter; and selectively supplying one or more fluids to or aspirating fluids from one or more of the first catheter, the second catheter, and the third catheter by the fluidics system.

Example 40. The method of any one of the preceding examples, but particularly Example 39, further comprising translating axially along the rail a fourth hub operatively coupled to a guidewire positioned within a third lumen of the third catheter.

Example 41. The method of any one of the preceding examples, but particularly Example 37, further comprising translating axially along the rail a third hub operatively coupled to a guidewire positioned within a second lumen of the second catheter.

Example 42. The method of any one of the preceding examples, but particularly Example 37, further comprising translating axially along the rail an introducer hub operatively coupled to a guide tube to position the guide tube in the access point vasculature, wherein the first catheter is positioned within a lumen of the guide tube.

Various systems and methods are described herein primarily in the context of a neurovascular access or procedure (e.g., neurothrombectomy). However, the catheters, systems (e.g., drive systems), and methods disclosed herein can be readily adapted for any of a wide variety of other diagnostic and therapeutic applications throughout the body, including particularly intravascular procedures such as in the peripheral vasculature (e.g., deep venous thrombosis), central vasculature (pulmonary embolism), and coronary vasculature, as well as procedures in other hollow organs or tubular structures in the body.

The systems and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor on the controller and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “arm” may include, and is contemplated to include, a plurality of arms. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A robotic medical system for performing a vascular procedure, comprising:
a procedure rail having a drive surface oriented along a generally vertical plane;
a rail adjustment system configured to couple to a patient support table and comprising one or more movable arm segments, the rail adjustment system configured to manipulate a position of the procedure rail relative to the patient support table;
a fluidics system; and
a plurality of catheter hubs operatively coupled to the procedure rail and configured to be axially translated along the drive surface along a longitudinal axis of the procedure rail;
wherein at least one of the plurality of catheter hubs is in fluid communication with the fluidics system, the at least one of the plurality of catheter hubs comprising a hemostasis valve and a catheter in communication with the hemostasis valve.

2. The robotic medical system of claim 1, wherein the fluidics system is configured to selectively supply fluid or vacuum to the hemostasis valve.

3. The robotic medical system of claim 2, wherein the fluidics system is configured to selectively supply saline from a saline source and contrast from a contrast source to the hemostasis valve.

4. The robotic medical system of claim 2, wherein the fluidics system comprises a cassette configured to receive saline from a saline source, receive contrast from a contrast source, and receive vacuum from a vacuum source.

5. The robotic medical system of claim 4, wherein the cassette comprises one or more robotically actuated valves configured to be controlled by a control system.

6. The robotic medical system of claim 2, further comprising:
a primary fluid line between the fluidics system and a junction point; and
a plurality of secondary fluid lines extending from the junction point, each of the plurality of secondary fluid lines coupled with one of the plurality of catheter hubs to supply fluid or vacuum thereto.

7. The robotic medical system of claim 6, wherein the junction point is coupled to the procedure rail.

8. The robotic medical system of claim 1, wherein the plurality of catheter hubs comprise:
at least one guidewire hub;
at least one guide catheter hub;
at least one access catheter hub; and
at least one procedure catheter hub.

9. The robotic medical system of claim 1, wherein, when in a procedure position, a proximal end of the procedure rail is positioned vertically above a distal end of the procedure rail, the distal end of the procedure rail being closer to a patient access point than the proximal end of the procedure rail.

10. The robotic medical system of claim 1, wherein the procedure rail is moveable from a retracted position to an extended position, wherein a distal end of the procedure rail is positioned closer to a patient access point in the extended position.

11. A method of performing a vascular procedure with a robotic medical system, the method comprising:
positioning a procedure rail via a rail adjustment system to approximately align a device stack profile to an access point vasculature of a patient, the procedure rail having a drive surface oriented along a generally vertical plane, the rail adjustment system coupled to a patient support table and comprising one or more movable arm segments;
translating a first hub operatively coupled to a first catheter axially along the drive surface of the procedure rail and toward the access point vasculature to introduce the first catheter into the access point vasculature;
translating a second hub operatively coupled to a second catheter axially along the drive surface of the procedure rail, the second catheter positioned within a first lumen of the first catheter; and
selectively supplying one or more fluids to or aspirating fluids from one or both of the first catheter and the second catheter by a fluidics system.

12. The method of claim 11, further comprising:
translating a third hub operatively coupled to a third catheter axially along the drive surface of the procedure rail, the third catheter positioned within a second lumen of the second catheter; and
selectively supplying one or more fluids to or aspirating fluids from one or more of the first catheter, the second catheter, and the third catheter by the fluidics system.

13. The method of claim 12, further comprising translating a fourth hub axially along the drive surface of the procedure rail, the fourth hub operatively coupled to a guidewire positioned within a third lumen of the third catheter.

14. The method of claim 11, further comprising translating a third hub axially along the drive surface of the procedure rail, the third hub operatively coupled to a guidewire positioned within a second lumen of the second catheter.

15. The method of claim 11, wherein selectively supplying one or more fluids to or aspirating fluids from one or both of the first catheter and the second catheter by the fluidics system comprises selectively supplying saline from a saline source and contrast from a contrast source to one or both of the first catheter and the second catheter.

16. The method of claim 11, wherein the fluidics system comprises a cassette configured to receive saline from a saline source, receive contrast from a contrast source, and receive vacuum from a vacuum source.

17. The method of claim 16, further comprising actuating one or more robotically actuated valves by a control system.

18. The method of claim 11, wherein the robotic medical system comprises:
a primary fluid line between the fluidics system and a junction point; and
a first secondary fluid line extending from the junction point to the first hub to supply fluid and vacuum thereto and a second secondary fluid line extending from the junction point to the second hub to supply fluid or vacuum thereto.

19. The method of claim 18, wherein the junction point is coupled to the procedure rail.

20. The method of claim 11, wherein, when the device stack profile is approximately aligned with the patient access point, a proximal end of the procedure rail is positioned vertically above a distal end of the procedure rail, the distal end of the procedure rail being closer to a patient access point than the proximal end of the procedure rail.
